SEQUENCE LISTING (22 sequences)

SEQ ID NO: 1 - DNA, 28 bp, Artificial Sequence
  Primer 1
ctcgaattcactctcttccgcatcgctg

SEQ ID NO: 2 - DNA, 29 bp, Artificial Sequence
  Primer 2
caggaattcttgcgactgtgactggttag

SEQ ID NO: 3 - DNA, 25 bp, Artificial Sequence
  Primer 3
gcgaaatggatttttgcatcgagct

SEQ ID NO: 4 - DNA, 48 bp, Artificial Sequence
  Primer 4
ggctctagacatatgtttattaatgatgatgatgatgatggtcgacgg

SEQ ID NO: 5 - DNA, 30 bp, Artificial Sequence
  Primer 5
agtggatcctacgagtgataatggagcatc

SEQ ID NO: 6 - DNA, 20 bp, Artificial Sequence
  Primer 6
ttgacaactgtttggagggc

SEQ ID NO: 7 - DNA, 20 bp, Artificial Sequence
  Primer 7
gctaactagagaacccactg

SEQ ID NO: 8 - DNA, 22 bp, Artificial Sequence
  Primer 8
taactagaggatccgataggcg

SEQ ID NO: 9 - DNA, 42 bp, Artificial Sequence
  Primer 9
cgacatatgtagatgcattagtttgtgttatgtttcaacgtg

SEQ ID NO: 10 - DNA, 19 bp, Artificial Sequence
  Primer 10
ggagaccactgccatgttg

SEQ ID NO: 11 - DNA, 28 bp, Artificial Sequence
  Primer 11
gccatccgcggcgagacctttgccacac

SEQ ID NO: 12 - DNA, 19 bp, Artificial Sequence
  Primer 12
tcactgacggtggtgatgg

SEQ ID NO: 13 - DNA, 20 bp, Artificial Sequence
  Primer 13
ggcagaagatcccctcgttg

SEQ ID NO: 14 - DNA, 28 bp, Artificial Sequence
  Primer 14
gtgtggcaaaggtctcgccgcggatggc

SEQ ID NO: 15 - DNA, 43 bp, Artificial Sequence
  Primer 15
tgctctagatcatatgcttattagtctaggcttagttctcttc

SEQ ID NO: 16 - DNA, 67 bp, Artificial Sequence
  Primer 16
catcccatggtccgcggtggaggtggatcaggtggaggtggctcagcaaacataaaactaagcgtac

SEQ ID NO: 17 - DNA, 20 bp, Artificial Sequence
  Primer 17
atgatgccgcagtggtctta

SEQ ID NO: 18 - DNA, 21 bp, Artificial Sequence
  Primer 18
gtcaaagtacgtggaagccat

SEQ ID NO: 19 - protein, 195 aa, Artificial Sequence
  Amino acid sequence of Tail-T(ii)-MH
MKRARPSEDTFNPVYPYDTETGPPTVPFLTPPFVSPNGFQESPPGVLSLRLSDPTSDNGASLSKGLESRVSALEKTSQIHSDTILRITQGLDDANKRIIALEQSRDDLVASVSDAQLAISRLESSIGALQTVVNGLDSSVTQLGARVGQLETGLADVRVDPGGGGSELGTKLGPEQKLISEEDLNSAVDHHHHHH

SEQ ID NO: 20 - protein, 269 aa, Artificial Sequence
  Amino acid sequence of Tail-T(ii)ev-Ang
MKRARPSEDTFNPVYPYDTETGPPTVPFLTPPFVSPNGFQESPPGVLSLRLSDPTSDNGASLSKGLESRVSALEKTSQIHSDTILRITQGLDDANKRIIALEQSRDDLVASVSDAQLAISRLESSIGALQTVVNGLDSSVTQLGARVGQLETGLADVRVDHDNLVARVDTAERNIGSLTTELSTLTLRVTSIQADFESRISTLERTAVTSAGAPLSIRNNRMTMVRGGGGSGGGGSANIKLSVQMKLFKRHLKWKIIVKLNDGRELSLD

SEQ ID NO: 21 - protein, 385 aa, Artificial Sequence
  Amino acid sequence of Tail-T(ii)ev-CD40L
MKRARPSEDTFNPVYPYDTETGPPTVPFLTPPFVSPNGFQESPPGVLSLRLSDPTSDNGASLSKGLESRVSALEKTSQIHSDTILRITQGLDDANKRIIALEQSRDDLVASVSDAQLAISRLESSIGALQTVVNGLDSSVTQLGARVGQLETGLADVRVDHDNLVARVDTAERNIGSLTTELSTLTLRVTSIQADFESRISTLERTAVTSAGAPLSIRNNRMTAGGGGSGGGGSGGGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 22 - protein, 6 aa, Artificial Sequence
  Protein conserved sequence
GVLXLX

(19) United States
(12) Patent Application Publication
(10) Pub. No.: US 2011/0189234 A1
(43) Pub. Date: Aug. 4, 2011

(54) ADENOVIRUS PARTICLES HAVING A CHIMERIC ADENOVIRUS SPIKE PROTEIN, USE THEREOF AND METHODS FOR PRODUCING SUCH PARTICLES

(76) Inventors: Victor Willem Van Beusechem, Amstelveen (NL); Frederik Hubertus Emanuel Schagen, Leiden (NL)

(21) Appl. No.: 12/279,114

(22) PCT Filed: Feb. 13, 2007
(86) PCT No.: PCT/NL07/50057

§ 371 Date: Feb. 2, 2009

(30) Foreign Application Priority Data
Feb. 13, 2006 (NL) PCT/NL2006/000072

(51) Int. Cl. A61K 39/235; C12N 7/01; C07H 21/04; C12N 15/861; A61P 35/00; A61P 37/02

(52) U.S. Cl. 424/233.1; 435/235.1; 536/23.72; 435/456

(57) ABSTRACT

The present invention is concerned with means and methods for producing adenovirus particles comprising a chimeric adenovirus spike protein that essentially lacks a functional knob domain. One aspect of the invention is concerned with a method for producing adenovirus particles comprising providing cells that are permissive for adenovirus replication with an adenovirus vector, with nucleic acid encoding said chimeric adenovirus spike protein and with nucleic acid encoding at least one adenovirus E3 region protein or a functional part, derivative and/or analogue thereof, said method further comprising culturing said permissive cells to allow for at least one replication cycle of said adenovirus virus and harvesting said adenovirus particle.

The invention relates to adenoviruses, adenovirus vectors and uses and methods of production thereof. The invention in particular relates to adenovirus particles comprising a fiber protein that lacks a fiber knob domain.

Human adenoviruses, in particular serotypes 2 and 5, are widely applied as vectors for gene delivery. These viruses have many potential therapeutic benefits, including easy propagation to high titers, efficient infection of dividing and non-dividing cells, and relatively limited toxicity in humans. However, the in vivo utility of adenovirus vectors (AdVs) is limited by their promiscuous tropism, which leads to efficient sequestration of administered AdVs in non-desired tissues, thereby limiting the fraction of the AdV dose available for target cell transduction. To overcome this limitation, strategies are being developed to redirect, i.e., “to target” entry of AdV to desired target cells. To accomplish this “targeting”, the native binding capacity of the AdV need to be abolished and the AdV need to be provided with a new binding affinity. The native tropism of adenovirus types 2 and 5 is defined by three physically distinct receptor-binding interactions. The primary attachment of adenovirus to host cells is mediated by an interaction of the C-terminal knob domain of adenovirus fiber with CAR [1-3]. A second receptor-binding site is localized to the penton base and mediates virus interaction with alpha v integrins [4-7]. A third receptor-binding site is localized to the third beta-spiral repeat in the fiber shaft and mediates binding to heparan sulphate glycosaminoglycans (HSG) [8,9]. Although CAR is the principal adenovirus attachment receptor, all three binding-sites contribute significantly to the tropism of adenovirus in vivo [10-13]. To improve the in vivo utility of AdV it is therefore preferred to remove as much as possible native binding sites from the virus capsid, where it is further preferred to remove all native binding sites.

The requirement for fiber in the interaction of adenovirus with host cells has directed most AdV targeting strategies to exploit this capsid protein as a portal for development of new cellular affinities (for reviews see [14,15]). Among these approaches, the one-component targeting strategy based on genetic modification of the fiber gene is the most well-defined and effective method of generating targeted vectors. Adenovirus fibers are trimeric proteins that consist of a globular C-terminal domain (the “knob” domain), a central fibrous shaft and an N-terminal part (the “tail” domain) that attaches to the viral capsid. In the presence of the globular C-terminal domain, which is necessary for correct trimerization, the shaft segment adopts a triple beta-spiral conformation. Fiber proteins are incorporated as trimers into the capsid structure. Genetic modification of the binding-specificity of the fiber has been accomplished in different ways. Addition of targeting epitopes to the C-terminus of fiber has been applied successfully but is limited to linear peptides of ˜20 to 25 residues [16-19]. Another approach is to incorporate inserts into the HI-loop of the fiber knob [20-22]. This site has been shown to tolerate introduction of certain peptides larger than 100 residues without substantially affecting propagation and infectivity of the resulting AdVs [23]. However, insertion of complexly folded and consequently more selective ligands appears to disturb trimerization of the fiber and prevent subsequent incorporation of fiber into the adenovirus capsid. To circumvent these constraints and broaden the range of targeting epitopes, recombinant spike molecules have been developed in which the fiber knob domain alone or in combination with (part of) the fiber shaft domain has been replaced with an exogenous trimerization domain and an exogenous receptor-binding moiety [24-26]. This approach has the additional advantage that it removes native binding sites residing in the fiber knob. Recombinant spike molecules are referred to herein as “knobless fibers” or “chimeric adenovirus spike proteins”. A knobless fiber molecule or chimeric adenovirus spike protein is defined in that it essentially lacks a functional fiber knob domain, is capable of forming trimers and is capable of attaching onto an adenovirus capsid. A “knobless fiber” does thus not mean that the molecule is a fiber protein lacking the knob domain. While this may be the case, other regions of the fiber, such as the shaft domain or part thereof, may also be lacking. A chimeric adenovirus spike protein of the invention may further comprise additional sequences such as targeting sequences and/or spacer/linker sequences. The “trimerization” domain of the fiber protein is, as mentioned, located in the knob domain. If the knob domain is removed from the fiber thereby creating a knobless fiber, it is preferred that the lost trimerization function is replaced by other sequences comprising a so-called “trimerization domain”. Otherwise, no trimers are formed and no fiber incorporated into the adenovirus particle. In the art different trimerization domains have been produced to replace the adenovirus trimerization domain. Heterologous trimerization domains can be derived from many different kinds of proteins. Non-limiting examples of knobless fiber proteins of the invention are described in WO01/81607, in WO01/02431 and in WO 98/54346.

The fiber “tail” domain provides the attachment function of the fiber to the adenovirus capsid. This attachment function is provided by a nuclear localization sequence, to transport the fiber to the nucleus where the adenovirus particles are assembled, and a recognition sequence for binding the fiber to penton base proteins in the adenovirus capsid. It is preferred that a knobless fiber of the invention comprises at least a functional part of this tail domain, where functional means providing capacity to bind to the adenovirus capsid when expressed in a cell. A knobless fiber of the present invention thus preferably comprises an adenovirus fiber “tail” domain and a heterologous and/or non-adenovirus trimerization domain. A knobless fiber of the invention preferably further comprises a heterologous targeting domain (or binding moiety). For means and method for producing knobless fiber containing adenoviruses reference is made to WO01/81607, which is incorporated by reference herein. Reference is also made to the examples of the present application. A heterologous trimerization domain is preferably derived from a viral protein, preferably derived from a non-enveloped virus. In a particularly preferred embodiment said trimerization domain comprises an oligomerization domain of a virus of the Reoviridae family or a functional part, derivative and/or analogue thereof. In a preferred embodiment said oligomerization domain is derived from reovirus attachment protein σ1 or a functional part, derivative and/or analogue thereof. A functional part in this respect means a part that initiates trimerization of said chimeric adenovirus spike protein in the intracellular milieu of a host cell infected with a virus of the invention to such extent that a sufficient proportion of said chimeric adenovirus spike protein adapts a trimeric form, where sufficient means that this leads to incorporation of said chimeric adenovirus spike protein in the adenovirus capsid of the invention. Reovirus σ1 trimerizes efficiently and shows remarkable structural and functional similarities with the adenovirus fiber [29]. The σ1 crystal structure reveals a fibrous tail and globular head, which closely resembles the structure, formed by the fiber shaft and knob domains, respectively. In addition, σ1 and fiber are similarly organized in the localization of several functional regions (FIG. 1). Notably, however, the two molecules differ in the location of their trimerization-determining region. In fiber, this region co-localizes with the main tropism-determining region to the knob domain, whereas in σ1 the trimerization and tropism-determining regions are localized to separate domains, i.e. the so-called T(ii) domain and the head domain, respectively. Since the trimerization domain of reovirus σ1 resides in the T(ii)-domain, a functional part of σ1 thus comprises at least part of the T(ii)-domain. Said part of the T(ii)-domain may be derived from a single reovirus serotype, but it may also comprise T(ii)-domain elements from different reovirus serotypes or reovirus mutants (Bassel-Duby et al., Nature, 315, 421-423, 1985; Cashdollar et al., Proc. Natl. Acad. Sci. USA, 82, 24-28, 1985; Nibert et al., J. Virol., 64, 2976-2989, 1990) that together initiate trimerization of said chimeric adenovirus spike protein according to the invention. The physical separation of functional regions of σ1 over different structural domains suggests that native reovirus tropism, which is mainly defined by an interaction of the head domain with the junction adhesion molecule-A (JAM-A), can be ablated by deletion of the head domain without affecting trimerization [30]. In support of this contention, replacement of the 334 C-terminal residues of σ1 with the 291-residue chloramphenicol acetyltransferase (CAT) protein resulted in a fusion protein that trimerized efficiently and was incorporated into the reovirus capsid [3]-33]. CAT enzymatic activity was preserved, suggesting that the fusion did not impose constraints on proper folding of the enzyme.

Useful trimerization domains for the invention, including that of reovirus comprised in the T(ii) domain, are characterized in that they comprise an amino acid sequence comprising heptad repeats in which apolar residues regularly occupy the first and fourth position of a heptad. Peptides comprising said heptad repeats adopt alpha-helical coils that form oligomers, so-called alpha-helical coiled-coils. The stability of the oligomers formed by the trimerization domain increases with an increased number of heptad repeats comprising apolar residues at the first and fourth position of the repeat. WO01/81607 teaches that a peptide comprising 4 heptad repeats forms trimeric alpha-helical coiled-coils. The coiled-coil regions of the three different reovirus serotypes and their alignment is given by Nibert et al (supra), included by reference herein. These regions comprise 21 to 22.5 heptad repeats forming approximately 41 to 44 alpha-helical coils in the different serotype σ1 proteins. The Tail-T(ii)-MH chimeric adenovirus spike protein of the present invention (see sequence depicted in FIG. 9) comprises 13 heptad repeats from the T(ii) domain of reovirus type 3 Dearing. This protein formed oligomers with sufficient efficiency to allow efficient incorporation of the protein into adenovirus capsids and efficient AdV propagation (see examples). The Tail-T(ii)ev-MH, the Tail-T(ii)ev-Ang (sequence depicted in FIG. 10) and Tail-T(ii)ev-CD40L (sequence depicted in FIG. 11) chimeric adenovirus spike proteins of the present invention comprise 21 heptad repeats from the T(ii) domain of reovirus type 3 Dearing. Chimeric adenovirus spike proteins with 21 heptad repeats formed oligomers with higher efficiency than Tail-T(ii)-MH as evidenced by the fact that Western blots prepared under non-denaturing conditions detected a mixture monomers and oligomers of Tail-T(ii)-MH, but only oligomers of Tail-T(ii)ev-Ang. Thus, the chimeric adenovirus spike proteins of the invention comprise a trimerization domain consisting of at least 4 heptad repeats, preferably at least 13 heptad repeats, more preferably at least 21 heptad repeats, where said heptad repeats are preferably derived from the reovirus σ1 T(ii) domain. A functional equivalent of a heptad repeat of a reovirus σ1T(ii) domain comprises at least the apolar residues at the first and fourth position of the repeat. As the sequence identity between serotypes in this region is limited (overall 14%), the equivalent preferably comprises at least 90% and more preferably at least 95% sequence identity with said heptad repeat.

In a preferred embodiment, the invention provides a chimeric adenovirus spike protein comprising an adenovirus tail domain and a heterologous trimerization domain forming alpha-helical coiled-coils. Preferably, a so-called hinge region that provides a highly flexible structure separates said tail domain and said trimerization domain. In this embodiment, it is preferred that said hinge region is derived from reovirus σ1 protein. A hinge region of reovirus σ1 protein comprises preferably between 7 to 10 amino acids predicted to form beta-turns. Such a hinge region is present in the carboxy-terminal region of the T(i) domain immediately adjacent to the T(ii) domain (Nibert et al., supra; Leone et al., Virology 182, 346-350, 1991). Thus, a chimeric adenovirus spike protein according to the invention preferably further comprises an amino-terminal adenovirus tail domain, followed by at least 7 amino acids of the carboxy-terminal region of the T(i) domain of reovirus σ1 protein, followed by a trimerization domain defined supra, preferably comprising at least 13 heptad repeats derived from the reovirus σ1 T(ii) domain, more preferably at least 21 heptad repeats derived from the reovirus σ1 T(ii) domain. The Tail-T(ii)-MH (sequence depicted in FIG. 9), Tail-T(ii)ev-MH, Tail-T(ii)ev-Ang (sequence depicted in FIG. 10) and Tail-T(ii)ev-CD40L (sequence depicted in FIG. 11) chimeric adenovirus spike proteins of the present invention comprise said hinge region of reovirus σ1 protein. In a preferred embodiment a chimeric adenovirus spike protein of the invention comprises at least the tail domain sequence of a fiber as depicted in FIG. 9, 10 or 11. More preferably, a chimeric adenovirus spike protein of the invention further comprises a hinge region as depicted in FIG. 9, 10 or 11. In a preferred embodiment a chimeric adenovirus spike protein of the invention comprises at least an amino acid sequence from 1 to and including 160 depicted in FIG. 9, 10 or 11, or a functional part, derivative and/or analogue thereof. In a further preferred embodiment a chimeric adenovirus spike protein of the invention comprises at least an amino acid sequence from 1 to and including 224 depicted in FIG. 10 or 11, or a functional part, derivative and/or analogue thereof. A derivative comprises the same functional parts in kind. A preferred derivative comprises at least 90% sequence identity to the indicated amino acids wherein said tail part is from a fiber protein of a different adenovirus or different adenovirus serotype. A further preferred derivative comprises at least 90% sequence identity to the indicated amino acids wherein said trimerization domain part is from a trimerization domain of a reovirus attachment protein σ1 of a different reovirus or different reovirus serotype. Preferably said sequence identity is at least 95%. In a further preferred embodiment a chimeric adenovirus spike protein of the invention comprises an amino acid sequence as depicted in FIG. 9, 10 or 11 or a functional part, derivative and/or analogue thereof. In several reported cases, artificial spike molecules trimerized efficiently and conferred new tropism to the AdV. Although these studies supported the feasibility of this strategy, the applicability of this approach has so far been limited by the impaired propagation efficiency of these vectors, which requires complementation with wild-type fiber or reintroduction of the fiber gene in the AdV genome for efficient vector production [24, 26-28; Magnusson et al., J. Gene Med., 4, 356-370, 2002). In many cases for instance for the preparation of clinical grade AdV batches for use in gene therapy procedures it is preferred to avoid said complementation with wild-type fiber or reintroduction of the fiber gene in the AdV genome. The limited propagation efficiency of previously constructed targeted AdV with chimeric adenovirus spike molecules thus seriously hampers the use of these vectors and has thus far precluded exploitation of this technology in virotherapy strategies using replication competent adenoviruses. Consequently, targeted replication competent adenoviruses comprising a chimeric adenovirus spike protein that are essentially lacking a functional fiber knob domain are not known in the art. For these reasons, there is a clear need to overcome said limited propagation efficiency, without complementation with wild-type fiber during the production process or reintroduction of the fiber gene in the AdV or replication competent adenovirus genome.

In the present invention it was realized that defective propagation of adenoviruses with chimeric adenovirus spike molecules lacking the fiber knob domain alone or in combination with the fiber shaft domain was the result of a lost cell lysis function provided by said fiber knob domain. The present invention provides a solution for this problem by complementing this lost function. Propagation was significantly improved when the cell for propagating the virus was provided with an adenovirus E3 protein. The invention therefore, in one aspect, provides a method for propagating an adenovirus with a chimeric fiber that essentially lacks a functional fiber knob domain, said method comprising providing a cell permissive for adenovirus replication with said adenovirus and a nucleic acid encoding an E3 protein and culturing said cells to allow propagation of said adenovirus. The invention further provides an adenovirus particle comprising nucleic acid derived from an adenovirus and a chimeric adenovirus spike protein, wherein said adenovirus particle and spike protein essentially lack a functional fiber knob domain and wherein said nucleic acid comprises at least one coding region for a protein of an adenovirus E3 region or a functional part, derivative and/or analogue of said E3 protein. These viruses propagate efficiently in cells that are permissive for adenovirus propagation. In a preferred embodiment said nucleic acid comprises the E3-region or a functional part, derivative and/or analogue thereof.

The adenovirus E3 region encodes a compendium of proteins that are expressed during various stages of the adenovirus life cycle. Recent reviews on E3 proteins can be consulted for a comprehensive description of these proteins and their actions in adenovirus-infected cells (Wold & Chinnadurai, 2000; Lichtenstein et al., 2004). Most E3 encoded proteins have been shown to subvert host immune defence mechanisms. Their actions include down-regulation of HLA-I complex and EGF receptor expression on the host cell membrane and inhibition of the TNF response in virus infected cells. The E3 gp 19K protein is localized in the ER membrane and binds the MHC class I heavy chain and prevents transport to the cell surface, where it would otherwise present adenovirus antigens to CTLs. This gene product, in addition, delays the expression of MHC I (Bennett et al., 1999). The E3 RID and 14.7K proteins inhibit pro-apoptotic pathways. Because E3 region proteins can help protect adenovirus-infected host cells against immune responses, it has been suggested to include the E3 region in adenovirus gene transfer vectors, with the purpose to prolong transgene expression (U.S. Pat. No. 6,100,086). Although these E3 proteins are thus important for effective adenovirus replication in a human body, where they prevent eradication of virus-infected cells by the host immune system, they were found dispensable for replication of the virus in tissue culture, where a host immune response is non-existent.

One of the E3 gene products has been termed the adenovirus death protein (ADP), since it facilitates late cytolysis of the infected cell (Tollefson et al., 1996). Consequently, adenoviruses carrying the E3 region were found more potent in killing host cells than adenoviruses lacking the E3 region (Yu et al., 1999). Apart from by using ADP, adenoviruses can also lyse their host cell by destructing the cytokeratin network through cytokeratin-18 cleavage (Chen et al., J. Virol. 67, 3507-3514, 1993) and by inducing p53-dependent or p53-independent apoptosis (Teodoro and Branton, J. Virol. 71, 1739-1746, 1997; Braithwaite and Russell, Apoptosis 6, 359-370, 2001). In fact, adenovirus serotype 46 relies solely on other mechanisms to kill its host, as it does not carry a gene encoding ADP (Reddy et al., Virus Res. 2005 Oct 18 (Epub ahead of print]). Thus, although E3 ADP is known to aid effective lysis of infected host cells, it was found dispensable for propagation of the virus in tissue culture, because adenoviruses have various alternative ways of lysing their host cell. In fact, the most important process for host cell lysis does not seem to be ADP-dependent, as it was reported that rapid lysis of adenovirus-infected cells was p53-dependent (Hall et al, Nature Med. 4(1998):1068-1072; Goodrum and Ornelles, J. Virol. 72(1998):9479-9490; Dix et al, Cancer Res. 60(2000):2666-2672).

It has also been suggested that the E3 ADP protein could be used to inhibit a deleterious effect of expressing a toxic gene on viral vector propagation in host cells. In case this is done to produce an adenoviral vector, it was reported that it is preferred to delete E3 ADP from the E3 region and insert it into the E1 or E4 region (WO99/41398). Taken together, several functions have been ascribed to proteins encoded by the E3 region. These functions only include functions of E3 region proteins in the context of adenoviruses comprising a functional fiber knob domain. It has not been recognized nor anticipated before that another function of the E3 region could become apparent in the context of an adenovirus that essentially lacks a functional fiber knob domain. Hence, until the present invention it was not known that the E3 region would not be dispensable for effective propagation of adenovirus that essentially lacks a functional fiber knob domain.

In the present invention, it was found that an adenovirus with a chimeric fiber that essentially lacks a functional fiber knob domain and that also essentially lacks a nucleic acid encoding an E3 protein is severely inhibited in its propagation in tissue culture. The propagation inhibition is presumably due to a reduced capacity to spread from an infected host cell to other cells. A control adenovirus that is identical to said adenovirus, except for that it comprises a fiber protein with a functional fiber knob domain propagated efficiently. An adenovirus according to the invention that has a fiber protein that essentially lacks a functional fiber knob domain and that is complemented by an E3 protein propagates in tissue culture with essentially similar efficiency as said control virus. Hence, whereas the E3 region is commonly regarded as dispensable for propagation of adenoviruses in tissue culture, the present invention shows that it is not dispensable for propagation of adenoviruses that have a fiber that essentially lacks a functional fiber knob domain. The present invention thus provides a previously not recognized or anticipated new function of the adenovirus E3 region that only becomes apparent if the adenovirus essentially lacks a functional fiber knob domain.

The invention therefore, in one aspect, provides a method for propagating an adenovirus with a chimeric fiber that essentially lacks a functional fiber knob domain, said method comprising providing a cell permissive for adenovirus replication with said adenovirus and a nucleic acid comprising the E3-region or a functional part, derivative and/or analogue thereof, or a nucleic acid encoding an E3 protein and culturing said cells to allow propagation of said adenovirus. The invention further provides an adenovirus particle comprising nucleic acid derived from an adenovirus and a chimeric adenovirus spike protein, wherein said adenovirus particle and spike protein essentially lack a functional fiber knob domain and wherein said nucleic acid comprises at least one coding region for a protein of an adenovirus E3 region or a functional part, derivative and/or analogue of said E3 protein. In a preferred embodiment said E3-region or at least one E3 region encoded protein comprises an ADP gene or a functional part, derivative and/or analogue thereof. A functional part, derivative and/or analogue of ADP comprises the same cytolytic effect in kind not necessarily in amount as ADP. These viruses according to the invention propagate efficiently in cells that are permissive for adenovirus propagation.

ADP exerts its cytolytic effect during adenovirus replication in any host cell that is susceptible to productive adenovirus replication. WO03/057892 teaches that in cells with a dysfunctional p53 tumor suppressor pathway, restoration of p53 function by exogenous p53 expression accelerates adenovirus-induced cytolysis. The cytolysis enhancement by p53 is observed in the presence or absence of ADP. Thus, although the mechanisms of p53-mediated cytolysis and ADP-mediated cytolysis are distinct, in cells with a dysfunctional p53 pathway, p53 is considered a functional analogue of ADP for the purpose of the invention. The present invention anticipates that propagating an adenovirus with a chimeric fiber that essentially lacks a functional fiber knob domain in cells with a dysfunctional p53 pathway can be made more efficient by expressing p53 from the genome of said adenovirus. The present invention thus provides a previously not recognized or anticipated new function of p53 that only becomes apparent if the adenovirus essentially lacks a functional fiber knob domain.

The invention therefore provides a new platform for genetically targeted AdVs that can be produced efficiently; and for genetically targeted replication competent adenoviruses that propagate efficiently in cells allowing adenovirus replication. In a preferred embodiment the platform utilizes a protein that is a fusion protein containing tail domain of adenovirus fiber and the T(ii) domain of reovirus σ1. Preferably, said tail domain and said T(ii) domain are separated by a hinge region, where it is preferred that said hinge region is derived from reovirus σ1 protein. This preferred chimeric adenovirus spike protein of the invention preferably lacks CAR- and HSG-binding-sites to diminish native AdV tropism and provides target binding-specificity through an incorporated binding moiety. Introduction of sequences encoding this fusion molecule into the AdV genome allows efficient propagation of the vector and results in high-titer vector production. The infection profile of the genetically targeted AdV is defined by the binding-moiety incorporated in the σ1-based fusion molecule.

Useful binding moieties for incorporation into the genetically targeted AdV according to the invention are well known in the art. The invention is not restricted in any way with regard to said binding moiety. When said binding moiety interferes with trimerization when linked close to said trimerization domain, it is preferred that a linker is inserted between said trimerization domain and said binding moiety. When said binding moiety requires intracellular processing to adopt its functional binding capacity, said intracellular processing should be compatible with the intracellular trafficking of said chimeric adenovirus spike protein towards the nucleus. Non-limiting examples of binding moieties include ligands for receptors, such as cytokines, including but not limited to epidermal growth factor, tumor necrosis factor, hepatocyte growth factor, vascular endothelial growth factor, Fas-ligand, TNF-related apoptosis-inducing ligand, CD40-ligand, insulin-like growth factor, basic fibroblast growth factor, folate, platelet-derived growth factor, transferrin, etcetera, or functional parts thereof. Other non-limiting examples of binding moieties include cell adhesion molecules, including but not limited to intercellular adhesion molecule-I, vascular cell adhesion molecule or carbonic anhydrase IX, or functional parts thereof. A functional part of a binding moiety means that said part is capable of binding with similar specificity, not necessarily with similar affinity as the complete binding moiety. Binding moieties may also be synthetic peptide molecules with a desired binding profile, such as, e.g., Anginex that binds activated endothelial cells. Further non-limiting examples of binding moieties include short peptides with binding specificity. Such molecules can be selected e.g. by phage display techniques known in the art. Examples of such peptides are peptides that include RGD or NGR amino acid sequences known to bind alpha-v integrins and CD13 molecules, respectively. Binding moieties can also be derived from antibodies. Particularly suited molecules derived from antibodies are so-called single-chain antibodies and single-domain antibodies originating from camels, dromedaries, vicunas, alpacas or llamas. Also particularly suited molecules derived from antibodies are so-called intrabodies, i.e., antibodies that exhibit binding specificity in an intracellular milieu. Antibodies and peptides from phage display libraries can in principle be selected with any binding specificity, also if the nature of their binding counterpart has not been characterized. It is to be understood, therefore, that the variety of useful binding moieties for incorporation in the chimeric adenovirus spike proteins of the invention is almost limitless.

In a preferred embodiment the invention provides chimeric adenovirus spike proteins comprising binding moieties comprising Anginex to target towards activated endothelial cells or CD40-ligand to target towards dendritic cells (example 10). In a preferred embodiment said chimeric adenovirus fiber protein comprises a targeting part comprising a targeting sequence comprising amino acids 239 and further of FIG. 10 or 242 and further of FIG. 11, or a functional part, derivative and/or analogue thereof.

An adenovirus particle of the invention preferably comprises a recombinant adenovirus vector. An adenovirus vector comprises nucleic acid that can be packaged into an adenovirus particle, such nucleic acid; typically, though not necessarily comprises two inverted terminal repeat sequences and an adenovirus packaging signal. Various types of adenovirus vectors have been generated. Several types are listed below, however, many variants, alternatives and combinations have been generated in the art. Minimal adenovirus vectors comprise two terminal repeat sequences, a packaging signal and a nucleic acid of interest. Pseudotyped adenovirus vectors such as adenovirus/adeno-associated virus chimeras only have to comprise an adenovirus packaging signal. Other types of vectors contain at least some of the adenovirus protein coding domains. Examples of such vectors are adenovirus vectors that have one or more deletions in or of an early region. Very popular are E1 and/or E4 deleted vectors and conditionally replicative adenoviruses (infra).

Needles to say that packaging of an adenovirus vector having one or more deletions of regions that are necessary for adenovirus propagation requires that the producing cell has all the necessary virus proteins available to it. In a wild type adenovirus, the nucleic acid coding for these virus specific proteins is present in the virus particle. A deletion that affects the expression of a protein that is necessary for particle formation can be complemented in trans. This is typically done by providing the cell with nucleic acid encoding said protein. This in trans complementation can be done by transiently providing the packaging cells with nucleic acid encoding the trans complementing factor. Preferably, the packaging cells are stably transformed with said nucleic acid. Many different cell lines have been generated that are stably transformed with nucleic acid encoding one or more E1 and/or E4 region encoded proteins or derivatives thereof. Such cell lines are used to complement recombinant adenovirus with the corresponding deletions. Structural proteins that form the capsid of the adenovirus particle are often serotype dependent although this not need always be the case. Serotype dependency in the case of fiber protein seems to be limited to the “tail” section that interacts with penton base proteins of the adenovirus capsid. Various chimeric fibers have been produced in the art and the general theme is that adenovirus particles with any chimeric fiber can be produced as long as the serotype of the “tail” matches that of the capsid proteins of the base. It is generally accepted that the conserved sequence G-V-L-(S/T)-L-(R/K) is the tail/shaft junction. The G is amino acid 44 or 45 of the fiber, dependent on the serotype en counts as the first amino acid of the shaft. A tail of an adenovirus fiber is thus typically 43 or 44 amino acids long. Most of the fiber tails are 44 amino acids, including the one of adenovirus 5. Fiber tails are typically well conserved between adenovirus serotypes. Some adenovirus serotypes are more alike than others. For instance, adenovirus 2 and 5 are very similar. Adenovirus 5 fibers match well with adenovirus 2 base and vice versa. Both of these viruses belong to subgroup C adenoviruses. Matching thus means that at least said tail and penton base are derived from adenovirus serotypes of the same subgroup. Preferably, said tail and said base are derived from the same serotype as this warrants efficient propagation of the viruses. Considering that adenovirus 2 and 5 are mostly used in the community it is preferred that adenovirus sequences are derived from adenovirus 2 and/or adenovirus 5.

Thus one aspect of the invention provides a method for preparing a composition comprising an adenovirus particle that comprises a chimeric adenovirus spike protein that essentially lacks a functional fiber knob domain, said method comprising providing cells that are permissive for adenovirus replication with an adenovirus vector; with nucleic acid encoding a chimeric adenovirus spike protein that lacks a functional fiber knob domain; and with nucleic acid comprising the E3-region or a functional part, derivative and/or analogue thereof or encoding at least one adenovirus E3 region protein or a functional part, derivative and/or analogue thereof, said method further comprising culturing said permissive cells to allow for at least one replication cycle of said adenovirus virus and harvesting said adenovirus particles. It will be clear from the above that for each adenovirus according to the invention the at least functional part of the fiber tail domain of the chimeric adenovirus spike protein of the invention matches with the penton base protein of the adenovirus particle of the invention. A chimeric adenovirus spike protein of the invention may be provided in trans by the adenovirus-producing cell. It is preferred that the nucleic acid that is packaged into the adenovirus particle comprises nucleic acid encoding said chimeric adenovirus spike protein. Thus, in a preferred embodiment said adenovirus particle further comprises an adenovirus vector comprising a nucleic acid encoding said chimeric adenovirus spike protein. In this way, propagation of the adenovirus is not dependent on cells that express said chimeric adenovirus spike protein. This embodiment is particularly useful for so-called replication competent adenoviruses that can replicate in any cell that is permissive for adenovirus propagation. In one aspect the invention thus provides a replication competent adenovirus comprising a chimeric adenovirus spike protein and a nucleic acid comprising the E3-region or a functional part, derivative and/or analogue thereof or encoding an E3 region encoded protein. Replication competent viruses have many uses. From a clinical perspective, replication competent viruses are of interest in for example cancer virotherapy.

A number of therapeutic uses of adenoviruses have now moved on to clinical trials and the first anti-cancer medicines based on recombinant adenoviruses are already registered products in China. Adenovirus-based therapies in use can be divided into at least five groups: (i) gene therapy, (ii) Gene-Directed Enzyme Prodrug Therapy, (iii) oncolytic virotherapy, (iv) vaccination, and (v) anti-angiogenesis therapy.

(i) Gene therapy. Two types of gene therapy approaches with recombinant adenoviruses can be discriminated. First, a loss-of-function mutation in cells can be complemented by introducing a nucleic acid sequence encoding the lost function into affected cells by means of a recombinant adenovirus vector. Second, a gain-of-function mutation in cells can be antagonized by introducing a nucleic acid sequence encoding a molecule capable of inhibiting the gained function or capable of inhibiting expression of the gained function into affected cells by means of a recombinant adenovirus vector. Gene therapy with recombinant AdV is useful for treating many different diseases. The appropriate target cells for treatment of the disease by gene delivery using the AdV depend on the nature of said disease. Usually, these are the diseased cells, but in some cases a disease can also be treated by gene delivery to healthy cells in a body. The latter is the case, e.g., when the product encoded by the gene is secreted by the healthy cells and can reach the diseased cells, or when gene delivery to healthy cells helps to counteract secondary effects of the disease, thus inhibiting symptoms of the disease. Gene therapy uses of AdV are well known in the art. Recombinant AdV find particular use for treating cancer. In cancer cells, non-limiting examples of loss-of-function mutations are deletions or missense mutations in genes encoding tumour suppressor proteins, such as for example p53 and p16. Mutations in the p53 gene that lead to loss of function have been implicated in the development of a wide variety of human tumours (Wills et al., 1994). To remedy this defect and to induce apoptosis in the tumour cells, a number of vectors incorporating wild-type p53 have been constructed. Clinical trials testing the efficacies of these vectors in the treatment of lung, head and neck and liver cancers are under way. A first recombinant AdV expressing human wild-type p53 (Gendicine) is registered in China for treatment of head and neck squamous cell carcinoma. In cancer cells, non-limiting examples of gain-of-function mutations are expression of oncogenes, such as for example myc or ras, and expression of p53 inhibitors such as for example MDM2, Parc, COP-1, Pirh2, or human papillomavirus encoded E6 protein. Non-limiting examples of molecules capable of inhibiting gain-of-function mutations include antisense ribonucleic acid molecules, dominant-negative mutant proteins, ribozymes and various small non-coding ribonucleic acid molecules capable of mediating the selective post-transcriptional gene silencing process of RNA interference. Said small non-coding ribonucleic acid molecules include, among others, short hairpin RNA molecules, microRNA molecules and their precursors, such as pre-miRNA and pri-miRNA molecules.

(ii) Gene-Directed Enzyme Prodrug Therapy (GDEPT). AdV can also be used to deliver molecules to cells that can aid in selective elimination of said cells. This is of particular use for treating diseases involving uncontrolled cell growth, such as cancer. In this strategy, AdV are used to deliver a prodrug convertase into the cancer cells and then a non-toxic drug is administered that can be converted into a cytotoxic agent by said prodrug convertase in situ (Crystal, 1999). The gene encoding the prodrug convertase is usually called a suicide gene. This type of therapy is generally referred to as suicide gene therapy or Gene-Directed Enzyme Prodrug Therapy (GDEPT). Non-limiting examples of GDEPT systems that have been used with AdV to deliver the suicide gene include the Herpes simplex virus thymidine kinase (HSV-tk) gene in combination with the prodrug ganciclovir (GCV), Cytosine deaminase (CD) with 5-fluorocytosine (Hirschowitz et al., 1995), and carboxylesterase (CE) with CPT-11 (Oosterhoff et al., Mol. Cancer Ther., 2, 765-771, 2003).

(iii) Oncolytic virotherapy. Replication competent viruses, in particular adenoviruses, are finding increasing utility for the treatment of cancer. In particular, so-called conditionally replicative adenoviruses (CRAds) have been developed to selectively replicate in and kill cancer cells. Such cancer-specific CRAds represent a novel and very promising class of anticancer agents (reviewed by Heise and Kim, J. Clin. Invest. 105(2000):847-851; Alemany et al., Nat. Biotech. 18(2000):723-727; Gomez-Navarro and Curiel, Lancet Oncol. 1(2000):148-158)). The tumor-selective replication of CRAds is preferably chieved through two alternative strategies. In a first strategy, the expression of an essential early adenovirus gene is controlled by a tumor-specific promoter (e.g., Rodriguez et al., Cancer Res. 57(1997):2559-2563; Hallenbeck et al., Hum. Gene Ther. 10(1999):1721-1733; Tsukuda et al., Cancer Res. 62(2002):3438-3447; Huang et al., Gene Ther. 10(2003):1241-1247; Cuevas et al., Cancer Res. 63(2003):6877-6884). A further strategy involves the introduction of mutations in viral genes to abrogate the interaction of the encoded RNA or protein products with cellular proteins, necessary to complete the viral life cycle in normal cells, but not in tumor cells (e.g., Bischoff et al., Science 274(1996):373-376; Fueyo et al., Oncogene 19(2000):2-12; Heise et al., Clin. Cancer Res. 6(2000):4908-4914; Shen et al., J. Virol. 75(2001: 4297-4307; Cascallo et al., Cancer Res. 63(2003):5544-5550). During their replication in tumor cells CRAds destroy cancer cells by inducing lysis, a process that is further referred to as “oncolysis”. Release of viral progeny from lysed cancer cells offers the potential to amplify CRAds in situ and to achieve lateral spread to neighbouring cells in a solid tumor, thus expanding the oncolytic effect. The restriction of CRAd replication to cancer cells dictates the safety of the agent, by preventing lysis of normal tissue cells. Currently, CRAd-based cancer treatments are already being evaluated in clinical trials (e.g., Nemunaitis et al., Cancer Res. 60(2000):6359-6366; Khuri et al., Nature Med. 6(2000):879-885; Habib et al., Hum. Gene Ther. 12(2001):219-226). A CRAd that is called H101 was recently registered as a medicine for head and neck cancer in China. Yet another strategy involves for instance tissue specific targeting. This selects for replication of the adenovirus in cells comprising the specific target. If the cells are tumor cells, selective replication occurs.

(iv) Vaccination. Recombinant adenoviruses are also being used to stimulate an immune response against cancer cells. This is usually done in either of two ways. In the first way, an AdV is used to express an immune stimulatory molecule, such as for example a cytokine or a heat shock protein in a tumor or in a prepared tumor vaccine. The goal of this treatment is to more effectively attract immune cells to the tumor or tumor vaccine or to more effectively present tumor antigens to the immune system. In a variation of this approach, the immune stimulatory molecule is expressed by a replication competent or conditionally replicative adenovirus that is capable of replicating in the tumor cells or tumor vaccine. This should result in an even more effective presentation of tumor antigens to immune cells, because tumor antigens are released from tumor cells through the oncolysis induced by the adenovirus and immune cells are attracted to the site of adenovirus replication. In the second way, an AdV is used to directly deliver nucleic acid encoding one or more tumor antigens to antigen presenting cells of the immune system, thus bypassing uptake of said antigens by said antigen presenting cells. In this regard, the so-called dendritic cells are particularly attractive targets to deliver said nucleic acid encoding tumor antigens.

(v) Anti-angiogenesis therapy. Recombinant adenoviruses are also being used to inhibit new blood vessel formation in tumor tissue, thereby inhibiting growth of the tumor. This is usually done by delivering a cytotoxic or growth inhibitory protein to blood vessel cells, in particular vascular endothelial cells. Said cytotoxic protein is a protein that causes direct or indirect death of the cell in which it is expressed. Indirect death can also mean death by GDEPT (infra). In this case, said cytotoxic protein is thus a prodrug convertase. It will be clear that for this use it is important that the cytotoxic protein is not delivered to other cells than the desired blood vessel cells, to prevent toxicity to said other cells. A growth inhibitory protein for this purpose can e.g. be an antagonist of a signalling pathway involved in endothelial cell growth, such as e.g., the VEGF or HGF/c-Met pathways.

For therapeutic uses of adenoviruses it is preferred to efficiently deliver the adenovirus to the diseased cells, to tumor blood vessel cells or to the antigen presenting cells in the body. It is therefore preferred to minimize sequestration of administered virus by non-target tissues. In some cases, it is also desired to prevent delivery of the virus to certain tissues, where the virus or the introduced nucleic acid sequences may have undesired side effects. Therefore, it is preferred to direct the adenovirus to the chosen target cells. This can be done by targeting cell entry via molecules that are more abundantly expressed on target cells than on non-target cells. Preferably, said molecules are not expressed on non-target cells at all. The adenovirus particles according to the present invention are particularly useful for this purpose. Thus in a preferred embodiment the invention provides an adenovirus particle that comprises a chimeric adenovirus spike protein that essentially lacks a functional fiber knob domain, comprising a nucleic acid comprising the E3-region or a functional part, derivative and/or analogue thereof or encoding an E3 region encoded protein and a nucleic acid encoding a therapeutic product capable of complementing in cells a loss-of-function mutation or a gain-of-function mutation. In a variation of this embodiment said adenovirus particle of the invention comprises nucleic acid encoding p53 or a functional part, derivative and/or analogue thereof. The term a functional part, derivative and/or analogue of a p53 protein refers to a functional part, derivative and/or analogue of a p53 protein that comprises the same tumour suppressive activity in kind not necessarily in amount as wild type p53. The adenovirus particles according to this embodiment are particularly useful for targeted delivery of said nucleic acid to diseased cells, such as e.g. cancer cells. This provides the possibility to complement said loss-of-function or gain-of-function mutation in diseased target cells, but not in healthy non-target cells. In another preferred embodiment the invention provides an adenovirus particle that comprises a chimeric adenovirus spike protein that essentially lacks a functional fiber knob domain, comprising a nucleic acid comprising the E3-region or a functional part, derivative and/or analogue thereof or encoding an E3 region encoded protein and a ‘suicide gene’. The adenovirus particles according to this embodiment are also very useful for targeted delivery of said nucleic acid to cancer cells. This provides the possibility to express said suicide gene in cancer cells, to effect selective elimination of said cancer cells. In yet another preferred embodiment the invention provides an adenovirus particle that comprises a chimeric adenovirus spike protein that essentially lacks a functional fiber knob domain, comprising a nucleic acid encoding one or more immune stimulatory molecules, such as cytokines or heat shock proteins and encoding an E3-region and/or an E3 region encoded protein. The adenovirus particles according to this embodiment are also very useful for targeted delivery of said nucleic acid to cancer cells. This provides the possibility to effectively attract immune cells to said cancer cells and to effectively present tumor antigens to immune cells, causing an immune response to cancer cells expressing said tumor antigens. In yet another preferred embodiment the invention provides an adenovirus particle that comprises a chimeric adenovirus spike protein that essentially lacks a functional fiber knob domain, comprising a nucleic acid encoding one or more tumor antigens and encoding an E3 region and/or E3 region encoded protein. The adenovirus particles according to this embodiment are very useful for targeted delivery of said nucleic acid to immune cells, in particular dendritic cells. A particularly useful binding moiety for incorporation into the adenovirus particles according to this embodiment is CD40 ligand or a functional part thereof. This embodiment provides the possibility to effectively express said tumor antigens in said immune cells, causing an immune response against tumor cells expressing said tumor antigens. In yet another preferred embodiment the invention provides an adenovirus particle that comprises a chimeric adenovirus spike protein that essentially lacks a functional fiber knob domain, comprising a nucleic acid encoding a replication competent adenovirus and encoding an E3 region and/or an E3 region encoded protein. In this embodiment, it is preferred that said replication competent adenovirus is adapted to enable preferential replication in transformed cells versus untransformed or normal cells and that said adenovirus is capable of effectively killing cancer cells. Said preferred replication can be achieved by any of the strategies used to construct CRAds (supra). Preferably, said adaptation comprises a nucleic acid comprising a coding region encoding an adenovirus E1A protein wherein said E1A protein comprises a mutation in at least part of the pRb-binding CR2 domain, preferably a deletion encompassing amino acids 122 to 129 (LTCHEAGF) of E1A. In a particularly preferred embodiment, said nucleic acid encoding a replication competent adenovirus and encoding an E3 region and/or an E3 region encoded protein furthermore encodes a molecule capable of augmenting the potency of said replication competent adenovirus to kill cancer cells. Non-limiting examples of such molecules include immune stimulating cytokines, GDEPT-mediating suicide genes, molecules capable of suppressing virus inhibitory molecules by RNA interference and oncolysis-enhancing molecules. Non-limiting examples of replication competent adenoviruses encoding oncolysis-enhancing molecules are disclosed in WO 03/057892, incorporated herein by reference. Adenovirus particles and/or vectors according to this embodiment are very useful for targeted delivery of said nucleic acid to cancer cells to effect selective destruction of said cancer cells by replication of said nucleic acid. In yet another preferred embodiment the invention provides an adenovirus particle that comprises a chimeric adenovirus spike protein that essentially lacks a functional fiber knob domain, comprising a nucleic acid encoding cytotoxic protein and encoding an E3 region and/or an E3 region encoded protein. Adenovirus particles according to this embodiment are useful for targeted delivery of said nucleic acid to vascular endothelial cells, in particular activated vascular endothelial cells, in particular activated vascular endothelial cells of the vasculature in a tumor. A particularly useful binding moiety for incorporation into the adenovirus particles according to this embodiment is Anginex or a peptide comprising more than one copy of the Anginex amino acid sequence. This embodiment provides the possibility to effectively express said cytotoxic protein in said activated vascular endothelial cells, causing destruction of said vascular endothelial cells.

In another aspect the invention provides a nucleic acid comprising a coding region for a chimeric adenovirus spike protein that essentially lacks a functional fiber knob domain and wherein said nucleic acid further comprises an E3 region and/or at least one coding region of an adenovirus E3 region or a functional part, derivative and/or analogue thereof. Said nucleic acid may advantageously be used in the generation and/or cloning of adenovirus vectors of the invention. In a preferred embodiment said nucleic acid comprises an adenovirus vector comprising said nucleic acid comprising a coding region for a chimeric adenovirus spike protein that essentially lacks a functional fiber knob domain and an E3 region and/or at least one coding region of an adenovirus E3 region or a functional part, derivative and/or analogue thereof. Thus the invention further provides an adenovirus vector coding for an adenovirus particle of the invention. The invention thus further provides a method for producing an adenovirus comprising providing a host cell that is permissive for replication of said adenovirus with an adenovirus particle according to the invention, or a nucleic acid comprising an adenovirus vector of the invention. The invention thus further provides an isolated and/or recombinant cell comprising a nucleic acid of the invention and or an adenovirus vector of the invention. Further provided is a method for providing nucleic acid to a cell comprising contacting said cell with an adenovirus virus particle according to the invention.

As mentioned above, the adenoviruses of the invention replicate well also in the absence of wild type fiber protein that contains an essentially functional knob domain. The latter was typically used to propagate knobless viruses to produce larger batch sizes. The present invention therefore further provides a composition comprising adenovirus particles wherein said adenovirus particles comprise chimeric adenovirus spike proteins that essentially lack a functional fiber knob domain and wherein said composition is essentially free of fiber protein that contains an essentially functional knob domain. It will be clear that a composition according to the invention provides advancement over previously available compositions, where it could not be excluded that in addition to chimeric adenovirus spike proteins that essentially lack a functional fiber knob domain said previously available compositions could also contain fiber protein that contains an essentially functional knob domain. The presence of such contaminating fiber protein is highly undesirable from a good manufacturing standpoint as well as from a targeting standpoint. Good manufacturing procedures require that the manufacturing process is controlled, reproducible and validated. Targeting requires that the tropism of the adenovirus for host cells be defined. The uncontrolled presence of an unknown amount of contaminating fiber protein obstructs both these requirements. In addition, propagation of a recombinant adenovirus comprising chimeric adenovirus spike proteins essentially lacking a fiber knob domain without the requirement for complementation with a nucleic acid encoding fiber protein avoids the risk of reintroducing the fiber knob domain encoding nucleic acid sequence into the genome of said recombinant adenovirus through recombination.

Another aspect of the high titers that can be produced using a method for propagating an adenovirus of the invention is that high titer batches can be generated starting from smaller number of cells and that less cycles of propagation are required to scale up production to reach a certain desired amount of virus. The yield of virus propagated according to the invention in a cell that is permissive for adenovirus replication is essentially similar as the yield of an adenovirus comprising a functional fiber knob domain propagated in the same type of cell and is substantially higher than the yield of an adenovirus essentially lacking a fiber knob domain and also lacking a functional E3 region in the same type of cell. Substantially higher in this respect means at least 3-times more, preferably at least 5-times more and more preferably at least 10-times more. It is to be understood that said substantially higher yield could be obtained at every individual propagation cycle. Thus, for example an at least 5-times higher yield during a scaling up procedure comprising 5 subsequent propagation cycles consisting of inoculating cells with adenovirus, allowing the virus to replicate in the cells and harvesting progeny virus from the cell, will yield more than 3,000-times more final virus product. It will be clear that this aspect of the invention provides economical benefit. Shorter production time, lower personnel cost, less host cells, less culture medium and smaller culture vessels are needed to produce a batch of virus of a desired size. Using a method for propagating an adenovirus of the invention will allow production of more virus batches per time and/or production of virus batches at lower cost.

The adenovirus particles of the invention can be purified and concentrated using methods known in the art, including but not limited to density gradient centrifugation, dialysis and column chromatography separation. The yield of adenovirus particles of the invention after such purification and concentration starting from a crude preparation of adenovirus particles and host cells is essentially not different from the yield of adenovirus particles produced using similar procedures and using cells comprising a functional fiber knob domain. However, in order to generate a purified composition of adenovirus particles essentially lacking a fiber knob domain and also lacking a functional E3 region the purification procedure should start with substantially more host cells to give the same yield. Following any purification procedure known in the art, co-purified contaminants, in particular host cell DNA, will be present in the purified composition. A purification procedure starting with substantially more host cells results in substantially more co-purified contaminants in the purified composition. In general, it is preferred to limit the amount of co-purified contaminants as much as possible. The invention thus further provides a purified composition comprising adenovirus particles wherein said adenovirus particles comprise chimeric adenovirus spike proteins that essentially lack a functional fiber knob domain and wherein said composition is essentially free of fiber protein that contains an essentially functional knob domain. Said purified composition can be made with similar effort and at similar cost as a purified composition comprising a functional fiber knob domain. A purified composition according to the invention has the important advantage that it comprises substantially less co-purified contaminants as a similarly produced purified composition that was made from a crude preparation of adenovirus particles and host cells essentially lacking a fiber knob domain and also lacking a functional E3 region protein.

Adenovirus particles have a tendency to bind to red blood cells. In particular to human red blood cells. This property is often undesired in a therapeutic setting as the association with red blood cells changes the (bio)distribution and bio(availability) of the administered adenovirus. Both phenomena typically affect the effective amount of adenovirus particle that can reach the intended target tissue. If the target is a target that is favoured by the RBC associated adenovirus this effect is desired, however, often the target is another tissue or cell type. It appears that the knob domain of an adenovirus fiber protein is important to this binding. Spike or fiber protein that lacks a functional knob domain has a strongly reduced binding capacity to RBC. It was found that also other parts of the adenovirus capsid do not significantly bind to RBC in the absence of a functional knob domain. Fibers and spike proteins of the invention are therefore suited to alter the (bio)distribution and/or bio(availability) of an administered adenovirus. They are also suited to increase the effective titer of an adenovirus for in vivo administration as less of the adenovirus is scavenged by the RBCs. In one aspect the present invention provides the use of a chimeric adenovirus spike protein that essentially lacks a functional knob domain and comprises an oligomerization domain of reovirus attachment protein σ1 or a functional part, derivative and/or analogue thereof, for producing an adenovirus particle. In a preferred embodiment said chimeric spike protein is used for producing an adenovirus particle that exhibits reduced binding to a red blood cell when compared to an adenovirus particle comprising a functional knob domain. In another aspect the invention provides the use of an oligomerization domain of reovirus attachment protein σ1 or a functional part, derivative and/or analogue thereof for producing an adenovirus particle that exhibits reduced binding to a red blood cell when compared to an adenovirus particle comprising a functional knob domain. Further provided is a composition comprising an adenovirus particle comprising a chimeric adenovirus spike protein that essentially lacks a functional knob domain and comprises an oligomerization domain of reovirus attachment protein σ1 or a functional part, derivative and/or analogue thereof, and a red blood cell. In this composition the RBC is essentially free of associated adenovirus. In a preferred embodiment said red blood cell is a human red blood cell. In a further aspect the present invention provides a method for avoiding binding of an adenovirus to red blood cells, said method comprising producing an adenovirus particle comprising a chimeric adenovirus spike protein that essentially lacks a functional knob domain and comprises an oligomerization domain of reovirus attachment protein σ1 or a functional part, derivative and/or analogue thereof and contacting said the produced adenovirus particle with a red blood cell. It is preferred that said produced adenovirus particle does not comprise fiber having a functional knob domain. In another aspect the present invention provides the use of a chimeric adenovirus spike protein that essentially lacks a functional knob domain and comprises a heterologous trimerization domain, for producing an adenovirus particle. In a preferred embodiment said chimeric spike protein is used for producing an adenovirus particle that exhibits reduced binding to a red blood cell when compared to an adenovirus particle comprising a functional knob domain. Further provided is a composition comprising an adenovirus particle comprising a chimeric adenovirus spike protein that essentially lacks a functional knob domain and comprises a heterologous trimerization domain preferably an oligomerization domain of reovirus attachment protein σ1 or a functional part, derivative and/or analogue thereof, and a red blood cell. In this composition the RBC is essentially free of associated adenovirus. In a preferred embodiment said red blood cell is a human red blood cell. In a further aspect the present invention provides a method for avoiding binding of an adenovirus to red blood cells, said method comprising producing an adenovirus particle comprising a chimeric adenovirus spike protein that essentially lacks a functional knob domain and comprises a heterologous trimerization domain and contacting said the produced adenovirus particle with a red blood cell. It is preferred that said produced adenovirus particle does not comprise fiber having a functional knob domain.

### EXAMPLES

### Example 1

### Design and Construction of σ1 Fusion Proteins

The success of genetically targeted AdVs relies on development of fiber-like molecules that are ablated for native binding and can incorporate large and complex ligands without loss of trimeric quaternary structure. The capacity of the reovirus σ1 protein to tolerate extensive modifications prompted us to design a fusion protein comprising key σ1 domains (FIG. 1). This σ1 fusion protein, designated Tail-T(ii)-MH (sequence depicted in FIG. 9), consists of the N-terminal 54 residues (tail domain) of fiber and parts of the T(i) and T(ii) domains of σ1. The fiber tail domain mediates transport of fiber into the nucleus and incorporation of the molecule into the adenovirus capsid. We reasoned that the σ1 domain included in Tail-T(ii)-MH would facilitate trimerization through the heptad repeat sequences of the T(ii) domain but lack interactions with reovirus receptor JAM-A and sialic acid. Thus, this construct is incapable of binding to all known reovirus receptors.

To redirect the σ1-fusion protein to a specific model receptor, we introduced six consecutive histidine residues (H) at the fusion protein C-terminus. The targeting peptide binds selectively to an artificial model receptor, consisting of an anti-His single chain antibody linked to the transmembrane domain of the platelet-derived growth factor receptor (HissFv.rec). Introduction of HissFv.rec into 293 cells (293.HissFv.rec) or CHO cells (CHO-αHis) results in surface expression of the receptor [34, 35]. The cell lines 293.HissFv.rec and CHO-αHis were kindly provided by Dr. J. T. Douglas (UAB, Birmingham, Ala., USA) and Dr. T. Nakamura (Mayo Clinic College of Medicine, Rochester, Minn., USA), respectively. We also introduced a Myc-epitope tag (M) adjacent to the His tag to facilitate detection of the fusion proteins. The resulting σ1-fusion protein with 6H is/myc-epitope thus serves as a prototype chimeric adenovirus spike protein according to the invention. The binding moiety can be simply replaced by another binding moiety to derive another chimeric adenovirus spike protein with a different binding specificity.

The Ad5 fiber expression construct pCMV.tpl.Fiber was generated using PCR. First, the Ad5 fiber gene was amplified using primers that flank the fiber-encoding sequence. The resulting 1.8 kb PCR product was blunted and cloned into EcoRV-digested pcDNA3 (Invitrogen, San Diego, Calif., USA) generating pCMV.Fiber. The tripartite leader (tpl) was amplified from pMad5 [42] using the primers 5′-CTCGAATTCACTCTCTTCCGCATCGCTG-3′ and 5′-CAGGAATTCTTGCGACTGTGACTGGTTAG-3′. The resulting 203 by PCR fragment was digested with EcoRI (underlined) and inserted into the unique EcoRI site of pCMV.Fiber between the cytomegalovirus promoter (CMV) and the fiber-encoding sequence.

A derivative of pCMV.tpl.Fiber, designated pCMV.tpl.Fiber.ΔSV40pA, was made by partial digestion with AflIII and digestion with SmaI, isolation of the 5894 by fragment, Klenow fill-in and re-circularisation.

Backbone plasmid pCMV-(B-)-TSFLC-MycHis was generated by digestion of pCMV-TSFLC [24] with EcoRV and KpnI, re-circularisation, and subsequent digestion with BamHI and XbaI for insertion of a BamHI- and XbaI-digested, 113 by PCR fragment that was amplified from pcDNA3.1(−)/Myc-His/LacZ (Invitrogen) using the primers 5′-GCGAAATGGATTTTTGCATCGAGCT-3′ and 5′-GGCTCTAGACATATGTTTATTAATGATGATGATGATGATGGTCGACGG-3′ that contained Myc- and His-tags and an NdeI (underlined) restriction site directly following the polyadenylation signal.

pCMV.tpl.Fiber.ΔSV40pA was used to generate the σ1 expression construct pCMV.tpl.Sigma1(T3D). First, the baculovirus transfer vector B9D4/6 (Chappell et al., J. Virol., 72, 8205-8213, 1998) was digested with SmaI and XbaI, which resulted in a 1449 by fragment containing the σ1 cDNA from reovirus T3D. This fragment was inserted in the 4075 by backbone of pCMV.tpl.Fiber.ΔSV40pA, which was obtained after digestion with PstI and XbaI and blunting the PstI-site with T4 DNA polymerase.

The chimeric adenovirus spike protein expression construct pCMV.tpl.Adtail-Sigma1 in which most of the σ1-anchoring domain T(i) was replaced with the adenovirus tail domain was generated in two steps. First, pCMV.tpl.Sigma1(T3D) was digested with BamHI and BspEI and the 5121 by fragment was isolated. To re-introduce the σ1 hinge domain, which was removed from this fragment, we amplified this region from pCMV.tpl.Sigma1(T3D) using the primers: 5′-AGTGGATCCTACGAGTGATAATGGAGCATC-′3 and 5′-TTGACAACTGTTTGGAGGGC-′3, digested the resulting 249 by fragment with BamHI and BspEI and inserted it in the BamHI- and BspEI-digested 5121-bp fragment, generating pCMV.Sigma1(T3D)DeltaT(i). Second, a nucleic acid fragment comprising the tpl and fiber tail domain was amplified from pCMV.tpl.Fiber using the primers: 5′-GCTAAC′TAGAGAACCCACTG-′3 and 5′-TAACTAGAGGATCCGATAGGCG-′3. The PCR-product of 525 bp was digested with BamHI and inserted into the unique BamHI site of pCMV.Sigma1(T3D)DeltaT(i), generating the expression construct pCMV.tpl.Adtail-Sigma1.

The Tail-T(ii)-MH chimeric adenovirus spike protein expression construct pCMV.tpl.Adtail-σ1T(ii)-MH was generated by digesting pCMV.tpl.Adtail-Sigma1 with Bell, Klenow fill-in, and redigestion with MfeI. The Tail-T(ii)-encoding 1.5 kb fragment was inserted between the blunted BspEI-end and sticky MfeI-end of the 4.7 kb backbone of pCMV-(B-)-TSFLC-MycHis.

Sequences of all inserts were confirmed by automated sequencing.

### Example 2

### Functional Characterization of the Tail-T(ii)-MH Fusion Protein

To enable functional characterization of the fusion attachment protein, the plasmids encoding Tail-T(ii)-MH and fiber were introduced into 293T cells by transient transfection using Lipofectamine Plus (Invitrogen Life Technologies, Breda, The Netherlands) according to the manufacturer's instructions. Following 48 h incubation to allow protein expression, cell lysates were prepared using reporter lysis buffer (Promega, Madison, Wis., USA), and lysates were either incubated at 95° C. for 5 min in denaturating sample buffer (62.5 mM Tris-HCl [pH 6.8], 10% glycerol, 2% SDS, and 2.5% (3-mercaptoethanol) or kept on ice in native sample buffer (62.5 mM Tris-HCl [pH 6.8], 10% glycerol, and 0.1% SDS). On the basis of protein content, 3 mg of total cell lysate was used in case of the Tail-T(ii)-MH samples, and 50 mg of cell lysate was used in case of the fiber samples. Samples were resolved by SDS-10% PAGE and transferred to PVDF membranes (Bio-Rad, Hercules, Calif., USA). Recombinant proteins were detected using the fiber tail-specific MAb Ab4 (Neomarkers, Fremont, Calif., USA) and visualized using chemiluminescence following incubation of the membranes with rabbit anti-mouse immunoglobulin G conjugated to horseradish peroxidase (RaM HRP; Dako, Glostrup, Denmark) and Lumilightplus (Roche, Almere, The Netherlands). Using denaturating conditions, the fusion protein Tail-T(ii)-MH appeared as a single species of the expected ˜22 kDa (FIG. 2A). Using nondenaturating conditions, a distinct fraction of the Tail-T(ii)-MH migrated as an oligomer, although most of the expressed protein was found in monomeric form. The apparent molecular weight of the Tail-T(ii)-MH oligomer was larger than expected for a homotrimer. This finding is analogous to the slower migration profile of trimerized fiber, which exhibits a larger apparent molecular weight as result of partial unfolding of the N-terminus [36].

To determine the intracellular distribution of Tail-T(ii)-MH, 293T cells were transfected with the fusion protein-encoding plasmid and imaged 48 h after transfection by immunofluorescence microscopy (FIG. 2B). Therefore, cells were fixed with methanol:acetone (1:1) and incubated with MAb Ab4 to detect fiber and fusion proteins, and with σ1 head-specific MAb 9BG5 [46] to detect σ1. Fluorescein-isothiocyanate-labeled rabbit anti-mouse immunoglobulin G (RaM-FITC; Dako, Glostrup, Denmark) was used as the secondary antibody. Nuclear DNA was stained using 1.2 ng/ml Hoechst 33342 (Sigma, St. Louis, Mo., USA). As anticipated, the parental σ1 and fiber proteins were detected in the cytoplasm and nucleus of transfected cells, respectively, in accordance with the intracellular compartments accommodating reovirus and adenovirus assembly. The Tail-T(ii)-MH molecule was found predominantly in the nucleus, which confirms that the nuclear localization signal residing in the fiber tail domain directed import of the fusion proteins into the nuclear compartment.

### Example 3

### Generation and Propagation of Genetically Targeted AdVs

To investigate whether the fusion proteins are incorporated into adenovirus particles and yield AdV with newly directed tropism, we replaced the fiber gene with sequences encoding Tail-T(ii)-MH in the genome of an AdV generating either pAdG.L.ΔE3.Tail-T(ii)-MH, which lacks the E3 region or pAdG.L.Tail-T(ii)-MH, which contains the E3 region. In case of pAdG.L.ΔE3.Tail-T(ii)-MH the fusion molecule-encoding sequence was released from the donor plasmid pCMV.tpl.Adtail-σ1T(ii)-MH with NdeI and cloned into NdeI-linearized pBr/Ad.BamRITR-PΔE3ΔFib. pBr/Ad.BamRITR-PΔE3ΔFib is a derivative of pBr/Ad.BamRITR-PΔE3 containing the BamHI released Ad5 sequence from pAdeasy-1, enclosing nucleotide 21562 until the 3′ end (He et al., Proc Natl Acad Sci USA, 95, 2509-2514, 1998), but lacks the fiber encoding sequences. pBr/Ad.BamRITR-PΔE3ΔFib was generated by digestion of pBr/Ad.BamRITR-PΔE3 with NdeI and Sse8387I and insertion of an NdeI- and Sse8387I-digested 2,200-bp PCR fragment, which was generated with primers 5′-CGACATATGTAGATGCATTAGTTTGTGTTATGTTTCAACGTG-′3 and 5′-GGAGACCACTGCCATGTTG-′3 and re-introduced the parts of the E4 region which were lost due to the NdeI and Sse8387I digestion. In case of pAdG.L.Tail-T(ii)-MH, the NdeI-digested fragment encoding the fusion molecule was cloned into NdeI-linearized pBr/Ad.BamRAFIB, which is generated similarly as pBr/Ad.BamRITR-PΔE3ΔFib, but still contains the E3 region (Havenga et al., J. Virol., 7, 3335-3342, 2001). The resulting constructs were used to introduce Tail-T(ii)-MH via recombination into pAdEasy-1 (He et al., Proc Natl Acad Sci USA, 95, 2509-2514, 1998)., which generated pAdEasy.ΔE3.Adtail-σ1T(ii)-MH and pAdEasy.Adtail-σ1T(ii)-MH, respectively. Subsequently, these constructs were recombined with pAdTrack.CMV.Luc, which was constructed by digestion of pABS.4-CMV-Luc [45] with XbaI and SwaI, isolation of the luciferase-encoding fragment, and insertion into the XbaI- and EcoRV-digested pAdTrack-CMV [44]. The recombination generated the full-length genome of the AdVs AdG.L.ΔE3.Tail-T(ii)-MH and AdG.L.Tail-T(ii)-MH respectively. Control vector AdG.L was obtained by recombination of pAdEasy-1 and pAdTrack.CMV.Luc. Thus, all vectors contain GFP and luciferase reporter genes in place of the E1 region. AdG.L contains the wild type fiber gene and lacks the E3 region. AdG.L.ΔE3.Tail-T(ii)-MH carries the Tail-T(ii)MH encoding sequences in place of the fiber gene and lacks the E3 region. AdG.L.Tail-T(ii)-MH also carries the Tail-T(ii)MH encoding sequences in place of the fiber gene, but has an intact E3 region. The resulting vectors without and with E3 region, i.e. pAdG.L.ΔE3.Tail-T(ii)-MH and pAdG.L.Tail-T(ii)-MH, were PacI-linearized and transfected into 293.HissFv.rec cells using Lipofectamine Plus (Invitrogen Life Technologies) according to the manufacturer's instructions. The resulting AdG.L.ΔE3.Tail-T(ii)-MH and AdG.L.Tail-T(ii)-MH virus progeny was propagated using 293.HissFv.rec cells. Generation and propagation of the control vector AdG.L were facilitated using the Ad5 E1-transformed human embryonic kidney cell line 293, which was purchased from the American Type Culture Collection. Although AdG.L.ΔE3.Tail-T(ii)-MH could be made (see below), this was very difficult and titers remained low, also after multiple propagation cycles. In contrast, AdG.L and AdG.L.Tail-T(ii)-MH were easily generated and expanded.

### Example 4

### Propagation Efficiency of Recombinant AdV with or without E3 Region, Lacking Fiber Proteins and Carrying Tail-T(ii)-MH Fusion Proteins

To analyse differences in propagation efficiency, we infected 293.HissFv.rec cells with either AdG.L.ΔE3.Tail-T(ii)-MH, AdG.L.Tail-T(ii)-MH or with the control virus AdG.L at an MOI of 0.01 or 0.1. Viral replication and spread was monitored over a period of 9 days by means of GFP expression. The AdVs AdG.L.ΔE3.Tail-T(ii)-MH and AdG.L showed large differences in GFP expression profiles (see FIG. 3, first two columns). Forty-eight hours post infection, all infected cells showed a bright GFP expression. Three days after infection all wells infected with AdG.L show spherical groups of approximately 30-60 cells with GFP expression, gradually growing in size and number over the next few days. The cells, infected with an MOI of 0.01 showed 15-20 of such structures 7 days post infection. In addition to these “spheres”, the typical “comet” structures were observed as soon as three days post infection in the cells, infected with AdG.L at an MOI of 0.1. Seven days post infection 5-7 of such structures were seen in the cells, infected with an MOI of 0.01. The cells infected with AdG.L.ΔE3.Tail-T(ii)-MH, on the other hand, were only able to generate a few very small sphere-like structures of approximately 10-20 cells in total. Seven days post infection twenty of these small sphere-like structures could be observed in the wells, infected with an MOI of 0.01. Even though these spheres did grow in size and number over the days, they developed much slower than their counterparts in the AdG.L-infected wells. The comet-like structures were never observed in wells that were infected with AdG.L.ΔE3.Tail-T(ii)-MH. Inefficient propagation of recombinant adenoviruses creates a problem for cost-effective manufacturing and severely limits utility of replication-competent variants of such adenoviruses for virotherapy purposes. To solve this problem, we constructed a targeted AdV according to the invention, carrying Tail-T(ii)-MH fusion proteins for targeted cell entry and an intact E3 region, i.e. AdG.L.Tail-T(ii)-MH. To analyse if the reintroduction of the E3 region in AdG.L.Tail-T(ii)-MH indeed showed improved propagation efficiency of the retargeted virus, we also assayed viral replication and spread of AdG.L.Tail-T(ii)-MH by means of GFP expression. As can be seen in FIG. 3, last column, the virus containing the chimeric adenovirus spike and the early 3 region (MG.L.Tail-T(ii)-MH), appeared to replicate much quicker than the original recombinant, lacking this region (AdG.L.ΔE3.Tail-T(ii)-MH). The GFP expression analysis showed that the E3+ virus has a replication speed and spreading pattern, which resembles that of the control virus AdG.L more than that of AdG.L.ΔE3.Tail-T(ii)-MH. The spread of the virus is not restricted to a small sphere of 10-20 cells, as with AdG.L.ΔE3.Tail-T(ii)-MH, but forms large, more elliptically shaped spheres of fifty to a hundred cells. Remarkably, the sphere-like structures formed by cells infected with AdG.L.Tail-T(ii)-MH appeared to be larger than the spheres formed by cells infected with AdG.L. From these observations we can conclude that the E3 region compensates for the loss of adenovirus lytic capacity resulting from the deletion of the fiber knob and shaft domains.

The propagation profile of the three viruses was also monitored by the luciferase expression in infected cells. In this experiment, 5×10⁴ 293.HissFv.rec cells were infected with either AdG.L.ΔE3.Tail-T(ii)-MH, or AdG.L at an MOI of 0.01 IU/cell. After 1 hr incubation, the infection mixture was replaced with fresh medium and luciferase expression was analysed at regular time intervals (FIG. 4A). During the first replication round (approximately 48 hr after infection), the three viruses expressed similar increasing amounts luciferase in infected cells, indicating that they were capable of replicating their DNA to multiple copies. However, thereafter luciferase expression levels increased in cell cultures infected with AdG.L or AdG.L.Tail-T(ii)-MH, whereas they did hardly change in cultures infected with AdG.L.ΔE3.Tail-T(ii)-MH. This indicated that in contrast to AdG.L.ΔE3.Tail-T(ii)-MH, AdG.L and AdG.L.Tail-T(ii)-MH lysed initially infected cells and their progeny spread to new host cells. The luciferase expression profiles of AdG.L and AdG.L.Tail-T(ii)-MH were quite similar over the entire propagation time span analysed, indicating that these two viruses spread with similar efficiency. To confirm the high propagation efficiency of AdG.L.Tail-T(ii)-MH, we performed a similar experiment in triplicate. 293.HissFv.rec cells were seeded at a density of 5×10⁴ cells/well in 96-well plates and infected at an MOI of 0.004 IU/cell with either AdG.L.Tail-T(ii)-MH or AdG.L. At various intervals over a period of 10 days, cells were lysed using 50 μl reporter lysis buffer (Promega), and luciferase activity was measured by chemiluminescence (Promega) using a Berthold luminometer (Berthold, Bad Wildbad, Germany) (FIG. 4B). During the observation interval, AdG.L and AdG.L.Tail-T(ii)-MH produced similar luciferase expression profiles, suggesting similar high propagation efficiencies.

### Example 5

### Reproducible Production of Purified High-Titer AdG.L.Tail-T(ii)-MH Batches

To assess the obtainable virus yield, crude virus stocks were generated at the scale of a T182 culture flask with helper cells. In case of AdG.L, we used 293 cells, while in case of AdG.L.Tail-T(ii)-MH and AdG.L.ΔE3.Tail-T(ii)-MH 293.HissFv.rec cells were used. After infection of the E1-complementing packaging cells, propagation was continued until the cells were in full CPE. For AdG.L.ΔE3.Tail-T(ii)-MH this took longer than for the other two viruses. Subsequently, cells were harvested, cracked by three freeze-thaw cycles and debris was removed by sedimentation at 4000 rpm for 5 min. The amount of AdV genomes was determined by quantitative PCR for the adenovirus hexon gene. As can be seen in table 1, the AdG.L.Tail-T(ii)-MH vector was generated at a genome-containing particle amount that closely approached that of the control virus with wild type fiber, whereas the E3-deleted virus was generated at an amount of virus particles that was approximately ten times lower.

In addition, three independent CsCl-purified preparations of AdG.L and AdG.L.Tail-T(ii)-MH were made according to standard techniques known in the art. Virus progeny was propagated up to the scale of twenty T182 flasks using 293.HissFv.rec cells in case of AdG.L.Tail-T(ii)-MH and 293 cells in case of AdG.L. The final virus harvests were purified by two successive rounds of CsCl centrifugation, dialysed against 10 mM Hepes pH 7.4, 10% glycerol, and 1 mM MgCl₂, and stored −80° C. The virus particle yield was determined by measuring the OD260 following denaturation of the virus in PBS, 1% SDS, and 1 mM EDTA (pH 8.0) at 55° C. These procedures reproducibly yielded similar quantities of viral particles (i.e., 10¹²-10¹³ genome-containing particles/twenty T182 flasks) of both AdVs.

### Example 6

### Characterization of AdG.L.Tail-T(ii)-MH Virions

To determine whether the Tail-T(ii)-MH attachment protein was incorporated onto the adenovirus capsid, we used SDS-PAGE to resolve the structural proteins of AdG.L.Tail-T(ii)-MH. An amount of 1.2×10¹¹ CsCl-purified particles of either AdG.L.Tail-T(ii)-MH or AdΔ24, a control adenovirus expressing wild-type fiber, were incubated at 95° C. for 5 min in denaturating sample buffer (62.5 mM Tris-HCl [pH 6.8], 10% glycerol, 2% SDS, and 2.5% β-mercaptoethanol) and resolved by SDS-10% PAGE. Coomassie blue staining of the viral proteins showed a similar protein composition for both vectors (FIG. 5A). However, AdG.L.Tail-T(ii)-MH contained an additional band, which likely represents the 22 kDa Tail-T(ii)-MH fusion protein. Since protein Ma and fiber show similar migration properties using these gel conditions, the absence of fiber in AdG.L.Tail-T(ii)-MH could not be confirmed using this assay. The incorporation of Tail-T(ii)-MH into virus particles was further investigated by immunoblotting. In this case 5×10⁹ virions of both vectors were denaturated and fractionated by SDS-10% PAGE as above. Viral proteins were transferred to PVDF membranes, incubated with fiber tail-specific monoclonal antibody (MAb) Ab4 or Myc-specific MAb 9E10 as the primary antibodies, and visualized using RaM-HRP (Dako) and Lumilightplus (Roche) (FIG. 5B). The anti-fiber tail MAb detected the 64 kDa wild-type fiber on control vector AdG.L particles and the 22 kDa Tail-T(ii)-MH chimeric adenovirus spike molecule on AdG.L.Tail-T(ii)-MH particles. Only the Tail-T(ii)-MH chimeric adenovirus spike protein was detected using a Myc-specific MAb, confirming that Tail-T(ii)-MH is efficiently and exclusively incorporated into the genetically modified AdV.

### Example 7

### Targeted Infectivity of AdG.L.Tail-T(ii)-MH

To assess the effect of removal of the fiber knob and shaft domains on the infectivity of the newly derived genetically targeted AdV, we compared the infectivity of AdG.L.Tail-T(ii)-MH to that of control vector AdG.L following adsorption to 293 cells and 293HissFv.rec cells, using both GFP expression (FIG. 6A) and luciferase activity (FIG. 6B) as readout. One day prior to infection, 293 and 293.HissFv.rec cells were seeded at a density of 5×10⁴ cells/well in 96 wells plates. The cells were infected with either vector at an MOI of 0.5 IU/cell for 2 h. Subsequently, infection mixtures were replaced with fresh medium. Two days after infection, GFP expression was assessed using fluorescence microscopy. Transduction efficiency of AdG.L.Tail-T(ii)-MH after infection of 293HissFv.rec cells was clearly enhanced in comparison to that following infection of 293 cells, while the transduction efficiency of AdG.L was similar after infection of both cell lines (FIG. 6A). Quantitation of this effect using luciferase expression showed that transduction efficiency by AdG.L.Tail-T(ii)-MH was about 40-fold greater after infection of 293HissFv.rec cells than after infection of 293 cells. Importantly, upon infection of 293 cells the de-targeting effect of AdG.L.Tail-T(ii)-MH resulted in a transduction efficiency, which was at least 35-fold lower than the AdG.L control vector

To confirm that transduction by the genetically targeted and control vectors was dependent on receptor-binding activities attributable to the respective attachment proteins we incubated both AdVs with either anti-knob MAb or anti-His-tag MAb prior to inoculation of 293.HissFv.rec or 293 cells (FIG. 7A). AdG.L.Tail-T(ii)-MH and AdG.L were incubated in the presence or absence of 300 ng anti-knob antibody (1D6.14) [47] or anti-His antibody (penta-His MAb; Qiagen, Hilden, Germany) at room temperature for 2 h. Pre-incubated mixtures were added to 293.HissFv.rec or 293 cells at an MOI of 0.5 IU/cell, incubated for 2 h and subsequently replaced with fresh medium. After 48 h incubation, cells were lysed, and luciferase activity was determined. Anti-knob MAb diminished transduction of both 293.HissFv.rec and 293 cells by AdG.L. In sharp contrast, anti-knob MAb had no effect on transduction of 293.HissFv.rec cells by AdG.L.Tail-T(ii)-MH. Conversely, anti-His-tag MAb did not affect transduction by AdG.L after infection of either cell type, but this MAb reduced AdG.L.Tail-T(ii)-MH transduction of 293HissFv.rec cells by approximately 90%. In addition, we analysed the infectivity of both AdVs on the Chinese hamster ovary cell line CHO (purchased from the ATCC) that lacks CAR expression; and on its derivative CHO-αHis that expresses the artificial scFv His-tag binding receptor. Both cell lines were seeded at a density of 2×10⁴ cells/well in 96-well plates. One day later, cells were incubated for 2 h with either AdG.L or AdG.L.Tail-T(ii)-MH at an MOI of 5 IU/cell. Forty-eight hours after infection the cells were lysed and luciferase activity was determined (FIG. 7B). As expected, transduction efficiency of AdG.L was similarly low on both cell lines. In contrast, AdG.L.Tail-T(ii)-MH exhibited a 12-fold increased transduction efficiency on CHO-αHis cells in comparison to CHO cells. Moreover, AdG.L.Tail-T(ii)-MH transduced the CHO-αHis cells significantly better than AdG.L. Together, these findings demonstrate that transduction by AdG.L.Tail-T(ii)-MH is principally defined by the Tail-T(ii)-MH protein and the artificial His-tag binding receptor.

### Example 8

### Construction, Generation and Propagation of a Recombinant AdV Lacking all Known Native Binding Sites and Comprising Tail-T(i)-MH Chimeric Adenovirus Spike Protein

The systemic applicability of AdV would be enhanced if transduction of non-desired tissues, most notably the liver, could be prevented. Although AdG.L.Tail-T(ii)-MH lacks all know reovirus and adenovirus binding-sites comprised in σ1 and fiber, respectively, the alpha v integrin binding site located in the adenovirus penton base protein is still present. To fully abolish the native adenovirus tropism, we also abolished this last known adenovirus receptor-interaction site. To this end, the integrin binding motif RGD in the penton base protein was changed into the non-binding motif RGE by site-directed mutagenesis of the penton base gene in the AdV genome using the primers 5′-GCCATCCGCGGCGAGACCTTTGCCACAC-′3, 5′-TCACTGACGGTGGTGATGG-′3, 5′-GGCAGAAGATCCCCTCGTTG-′3 and 5′-GTGTGGCAAAGGTCTCGCCGCGGATGGC-′3, and pBHG11 (Bett et al. Proc. Natl. Acad. Sci. USA, 91, 8802-8806, 1994) as template. The resulting PCR product containing the mutated (and thus now inactivated) integrin-binding site, designated as p*, was digested with PmeI and AscI and inserted in pBHG11ΔAsc. This derivative of pBHG11 was generated by digestion of pBHG11 with AscI and religation. After insertion of p* into pBHG11ΔAsc, the initially removed AscI fragment was re-introduced, forming pBHG11P*. This construct was digested with RsrII and the penton base gene-containing fragment of 7707 by was isolated and inserted into the 27,246 bp, RsrII-digested fragment of pAdEasy.Adtail-σ1T(ii)-MH. The resulting, so-called pAdEasy.p*.Adtail-σ1T(ii)-MH construct was recombined with pAdTrack.CMV.Luc to generate pAdG.L.p*.Tail-T(ii)-MH. Thus, this construct contains a adenovirus full-length genome with GFP and luciferase reporter genes in place of the E1 region, the complete E3 region and the Tail-T(ii)-MH-encoding sequences in place of the fiber gene. In addition, it lacks the integrin-binding site by mutation of the RGD motif residing in penton base protein. To generate the AdV AdG.L.p*.Tail-T(ii)-MH, the construct pAdG.L.p*.Tail-T(ii)-MH was PacI-linearized and transfected into 293.HissFv.rec cells using Lipofectamine Plus (Invitrogen Life Technologies) according to the manufacturer's instructions. The resulting virus progeny was propagated using 293.HissFv.rec cells. Propagation of this AdV up to the scale of twenty T182 flasks and subsequent purification by two successive rounds of CsCl centrifugation and dialysis, according to standard techniques known in the art, yielded a composition comprising a high quantity of virus particles, i.e. 7×10¹² genome-containing particles/twenty T182 flasks.

### Example 9

### Biodistribution and Retention in the Circulation of AdG.L.p*.Tail-T(ii)-MH Virus After Intravenous Injection into Mice

To study the in vivo performance of AdG.L.p*Tail-T(ii)-MH, which is completely ablated for adenovirus and reovirus native tropism, we injected 1E+10 virus particles (vp) in the tail vein of C57bl/6 mice and analysed the transduction of tissues and persistence of the virus particles in the circulation. We collected small blood samples at 2, 5, 10, 20, 30, 60 and 120 minutes after injection for analysis. Forty-eight hrs after administration, the mice were sacrificed and liver, spleen, heart, lungs and kidneys were isolated, frozen immediately in liquid nitrogen and homogenized by grinding. Lysates of ground tissues were prepared and luciferase expression was measured by chemiluminescence (Promega) using a Berthold luminometer (Berthold). The lysates were normalized for protein content as was determined by Bradford assay (Bio-Rad), using bovine serum albumin as standard. In all analysed tissues we observed a significant reduction of transduction by AdG.L.p*Tail-T(ii)-MH in comparison to the control AdG.L (p<0.01). Moreover, AdG.L.p*Tail-T(ii)-MH did not show any transduction of lung and kidneys in sharp contrast to the control vector. Analysis of infectious virus in the blood was performed by using 2 μl of the obtained blood samples for infection of 293.HissFv.rec cells. Two days after infection cells were lysed and luciferase activity was determined. Also in this assay, AdG.L.p*Tail-T(ii)-MH showed an improved in vivo performance (see FIG. 8). Whereas AdG.L was readily cleared from the blood, leaving less than 1% of the administered dose after 10 minutes and declined to less than 0.1% after 30 minutes, AdG.L.p*Tail-T(ii)-MH declined to 1% of the administered dose only after 1 hour, and this level remained stable for at least another hour.

### Example 10

### Construction and Analysis of Chimeric Adenovirus Spike Proteins with an Extended Reovirus σ1 T(ii) Domain Comprising Anginex or CD40-Ligand Binding Moieties

The Tail-T(ii)ev-MH chimeric adenovirus spike protein expression construct pCMV.tpl.Adtail-σ1T(ii)ev-MH was made as follows. First, the T(ii)ev encoding domain was isolated from pCMV.tpl.Adtail-Sigma1(T3D) by digesting with NcoI, Klenow fill-in and re-digestion with AgeI. The resulting 607-bp fragment was inserted in a pCMV.tpl.Adtail-σ1T(ii)-MH-derived backbone of 5840 bp, which was isolated after digestion of pCMV.tpl.Adtail-σ1T(ii)-MH with BsiWI, Klenow-fill-in and redigestion with AgeI. pCMV.tpl.Adtail-σ1T(ii)ev-MH differs from pCMV.tpl.Adtail-σ1T(ii)-MH in that it comprises a larger part of the reovirus σ1T(ii) domain comprising 21 in stead of 13 heptad repeats. Next, we constructed the new chimeric adenovirus spike protein expression construct pCMV.tpl.Adtail-σ1T(ii)ev-Ang encoding Tail-T(ii)ev-Ang chimeric adenovirus spike protein comprising an Anginex binding moiety. To generate pCMV.tpl.Adtail-σ1T(ii)ev-Ang, we amplified the Anginex encoding sequence (Griffioen et al., Biochem. J., 354, 233-242, 2001) using the primers: 5′-TGC TCT AGA TCA TAT GCT TAT TAG TCT AGG CTT AGT TCT CTT C-′3 and 5′-CAT CCC ATG GTC CGC GGT GGA GGT GGA TCA GGT GGA GGT GGC TCA GCA AAC ATA AAA CTA AGC GTA C-′3 and digested the resulting 167-bp PCR fragment with XbaI and NcoI (underlined). The resulting fragment was ligated with a 964 by fragment, which was isolated after digestion of pCMV.tpl.Adtail-Sigma1(T3D) with HindIII and NcoI, and a 5352 by fragment, which was isolated after digestion of pCMV.tpl.Adtail-σ1T(ii)-MH with HindIII and XbaI. A sequence of this chimeric adenovirus spike protein is given in FIG. 10. Tail-T(ii)ev-Ang chimeric spike protein was expressed and analysed by Western blot as described for Tail-T(ii)-MH in example 2. This revealed that Tail-T(ii)ev-Ang, in contrast to Tail-T(ii)-MH, was found exclusively as oligomers, showing that oligomerization by Tail-T(ii)ev-Ang was more efficient than that of Tail-T(ii)-MH. We contributed this to the larger number of heptad repeats in Tail-T(ii)ev-Ang compared to Tail-T(ii)-MH. Based on this finding, we also constructed another chimeric adenovirus spike protein expression construct, designated pCMV.tpl.Adtail-σ1T(ii)ev-CD40L encoding Tail-T(ii)ev-CD40L chimeric adenovirus spike proteins comprising a CD40-ligand binding moiety. To generate pCMV.tpl.Adtail-σ1T(ii)ev-CD40L, we isolated the CD40L encoding sequence from pKan.FF/CD40L comprising the FF/CD40L fusion gene (Belousova et al., J. Virol., 77, 11367-11377, 2003) by digestion with NaeI and MfeI and Klenow fill-in. This blunted fragment of 532 by was ligated into the 6316-bp backbone of pCMV.tpl.Adtail-σ1T(ii)ev-MH, which was obtained after digestion with XbaI and Klenow fill-in, followed by partial digestion with NcoI and subsequent blunting. A sequence of this chimeric adenovirus spike protein is given in FIG. 11. Tail-T(ii)ev-CD40L was expressed as described in example 2 and shown to bind efficiently to cells expressing CD40, but not to control cells not expressing CD40, by FACS analysis.

### Example 11

### AdG.L.Tail-T(ii)-MH and AdG.L.p*.Tail-T(ii)-MH do not Bind to Human Red Blood Cells

AdVs with native tropism were reported to bind and agglutinate red blood cells of rat and human origin, but not mouse erythrocytes (Cichon et al., 2003; Nickol et al., 2004; Lyons et al., 2006). Obviously, the interaction with human erythrocytes forms a major hurdle for therapeutic application of AdVs. In addition to sequestration by the liver, AdV can also be sequestered by red blood cells in the human circulation. This aspect of AdV tropism with importance for systemic AdV administration cannot be studied in mice. Translation of the observed improved AdV bioavailability in the circulation of mice (Example 9) to the human situation required additional experiments with human erythrocytes. Therefore, we tested AdG.L.Tail-T(ii)-MH and AdG.L.p*.Tail-T(ii)-MH in comparison to native AdG.L for red blood cell binding and hemagglutination properties in vitro.

Fresh blood of mice, rats or humans was collected in EDTA-tubes and mixed with 1 volume equivalent Alsever solution (23 mM Tri-sodium citrate, 114 mM Glucose, 55 mM NaCl and 3 mM Citric acid; pH 6.1). Cells were sedimented at 1,200 g for 10 min and washed 3 times by repeated resuspension in 2 volume equivalents Alsever solution and centrifugation at 1,200 g for 10 min. Finally, the pellet was resuspended in Alsever solution to generate a 30% packed-cell suspension.

Testing for haemagglutination was performed with a 1% erythrocyte suspension, which was generated by dilution of the 30% packed-cell suspension in HA-buffer (PBS, 0.005% BSA). A volume of 50 μl 1% erythrocyte suspension was prelayed in the wells of a concave-bottom-shaped 96-well plate and gently mixed with 500 of a dilution series of each AdV (stock concentration: 1.0×10¹² vp/ml). After 2 h gravitational sedimentation, plates were photographed and analysed for haemagglutination characteristics of each AdV. Hemagglutination of human erythrocytes is shown in FIG. 12A. As can be seen, native AdG.L agglutinated human erythrocytes. It did also agglutinate rat erythrocytes, but not mouse erythrocytes (not shown). Importantly, neither AdG.L.Tail-T(ii)-MH nor AdG.L.p*Tail-T(ii)-MH agglutinated any of the red blood cell species (human RBC, FIG. 12A; rat and mouse RBC, not shown).

To corroborate these findings, we analyzed direct association of virus particles with human blood cells by determining the number of viral genomes bound to these cells using real-time PCR. A 30% packed-cell suspension of human blood cells prepared as above was diluted in PBS to a physiological concentration of 8.4×10⁸ erythrocytes per 250 μl. A total of 8.4×10⁷ virus particles was added and incubated for 60 min at 37° C. Next, virus particles bound to erythrocytes were separated from unbound virus by centrifugation at 1,200 g for 14 min. The erythrocyte pellet was washed twice with 10 volume equivalents PBS. Adenovirus DNA in bound and unbound virus fractions was isolated with the QIAamp DNA Blood Mini Kit (Qiagen), according to the manufacturer's protocol. The amount of viral genomes present was quantified in the LightCycler® 480 (Roche Diagnostics, Mannheim, Germany) using the LightCycler® 480 SYBR Green I Master kit, 20 pmol of forward hexon primer 5′-ATGATGCCGCAGTGGTCTTA-′3 and 20 pmol of reverse hexon primer 5′-GTCAAAGTACGTGGAAGCCAT-′3. A standard curve was generated with 10-fold serial dilutions of adenovirus DNA. As can be seen in FIG. 12B, AdG.L with native tropism bound human red blood cells efficiently, leaving less than 5% of the added virus dose unbound. In sharp contrast, the affinity of AdG.L.Tail-T(ii)-MH and AdG.L.p*Tail-T(ii)-MH for human red blood cells was clearly reduced. Less than 10% of these viruses was recovered from the red blood cell fraction.

Taken together, these data show that targeted AdV carrying Tail-T(ii)-MH attachment molecules evaded potential sequestration by human erythrocytes, suggesting that this type of targeted AdV might exhibit similarly extended circulation in the bloodstream of humans as was observed in mice.

### REFERENCES

1. Law, L. K. and Davidson, B. L. (2005) What does it take to bind CAR? Mol Ther, 12: 599-609.
2. Kirby, I., et al. (2000) Identification of contact residues and definition of the CAR-binding site of adenovirus type 5 fiber protein. J Virol, 74: 2804-2813.
3. Roelvink, P. W., Mi Lee, G., Einfeld, D. A., Kovesdi, I. and Wickham, T. J. (1999) Identification of a conserved receptor-binding site on the fiber proteins of CAR-recognizing adenoviridae. Science, 286: 1568-1571.
4. Mathias, P., Wickham, T., Moore, M. and Nemerow, G. (1994) Multiple adenovirus serotypes use alpha v integrins for infection. J Virol, 68: 6811-6814.
5. Li, E., et al. (2001) Integrin alpha(v) beta1 is an adenovirus coreceptor. J Virol, 75: 5405-5409.
6. Wickham, T. J., Filardo, E. J., Cheresh, D. A. and Nemerow, G. R. (1994) Integrin alpha v beta 5 selectively promotes adenovirus mediated cell membrane permeabilization. J Cell Biol, 127: 257-264.
7. Wickham, T. J., Mathias, P., Cheresh, D. A. and Nemerow, G. R. (1993) Integrins alpha v beta 3 and alpha v beta 5 promote adenovirus internalization but not virus attachment. Cell, 73: 309-319.
8. Dechecchi, M. C., et al. (2001) Heparan sulfate glycosaminoglycans are receptors sufficient to mediate the initial binding of adenovirus types 2 and 5. J Viral, 75: 8772-8780.
9. Dechecchi, M. C., Tamanini, A., Bonizzato, A. and Cabrini, G. (2000) Heparan sulfate glycosaminoglycans are involved in adenovirus type 5 and 2-host cell interactions. Virology, 268: 382-390.
10. Koizumi, N., et al. (2003) Reduction of natural adenovirus tropism to mouse liver by fiber-shaft exchange in combination with both CAR- and alphav integrin-binding ablation. J Virol, 77: 13062-13072.
11. Smith, T. A., et al. (2003) Adenovirus serotype 5 fiber shaft influences in vivo gene transfer in mice. Hum Gene Ther, 14: 777-787.
12. Nicklin, S. A., Wu, E., Nemerow, G. R. and Baker, A.H. (2005) The influence of adenovirus fiber structure and function on vector development for gene therapy. Mol Ther, 12: 384-393.
13. Einfeld, D. A., et al. (2001) Reducing the native tropism of adenovirus vectors requires removal of both CAR and integrin interactions. J Virol, 75: 11284-11291.
14. Krasnykh, V. N., Douglas, J. T. and van Beusechem, V. W. (2000) Genetic targeting of adenoviral vectors. Mol Ther, 1: 391-405.
15. Barnett, B. G., Crews, C. J. and Douglas, J. T. (2002) Targeted adenoviral vectors. Biochim Biophys Acta, 1575: 1-14.
16. Michael, S. I., Hong, J. S., Curiel, D. T. and Engler, J. A. (1995) Addition of a short peptide ligand to the adenovirus fiber protein. Gene Ther, 2: 660-668.
17. Wickham, T. J., et al. (1997) Increased in vitro and in vivo gene transfer by adenovirus vectors containing chimeric fiber proteins. J Virol, 71: 8221-8229.
18. Hong, J. S. and Engler, J. A. (1996) Domains required for assembly of adenovirus type 2 fiber trimers. J Virol, 70: 7071-7078.
19. Wickham, T. J., Roelvink, P. W., Brough, D. E. and Kovesdi, I. (1996) Adenovirus targeted to heparan-containing receptors increases its gene delivery efficiency to multiple cell types. Nat Biotechnol, 14: 1570-1573.
20. Nicklin, S. A., et al. (2001) Ablating adenovirus type 5 fiber-CAR binding and HI loop insertion of the SIGYPLP peptide generate an endothelial cell-selective adenovirus. Mol Ther, 4: 534-542.
21. Krasnykh, V., et al. (1998) Characterization of an adenovirus vector containing a heterologous peptide epitope in the HI loop of the fiber knob. J Virol, 72: 1844-1852.
22. Dmitriev, I., et al. (1998) An adenovirus vector with genetically modified fibers demonstrates expanded tropism via utilization of a coxsackievirus and adenovirus receptor-independent cell entry mechanism. J Virol, 72: 9706-9713.
23. Belousova, N., Krendelchtchikova, V., Curiel, D. T. and Krasnykh, V. (2002) Modulation of adenovirus vector tropism via incorporation of polypeptide ligands into the fiber protein. J Virol, 76: 8621-8631.
24. van Beusechem, V. W., et al. (2000) Recombinant adenovirus vectors with knobless fibers for targeted gene transfer. Gene Ther, 7: 1940-1946.
25. Magnusson, Hong, S. S., Boulanger, P. and Lindholm, L. (2001) Genetic retargeting of adenovirus: novel strategy employing “deknobbing” of the fiber. J Virol, 75: 7280-7289.
26. Krasnykh, V., Belousova, N., Korokhov, N., Mikheeva, G. and Curiel, D. T. (2001) Genetic targeting of an adenovirus vector via replacement of the fiber protein with the phage T4 fibritin. J Virol, 75: 4176-4183.
27. Pereboeva, L., Komarova, S., Mahasreshti, P. J. and Curiel, D. T. (2004) Fiber-mosaic adenovirus as a novel approach to design genetically modified adenoviral vectors. Virus Res, 105: 35-46.
28. Belousova, N., et al. (2003) Genetically targeted adenovirus vector directed to CD40-expressing cells. J Virol, 77: 11367-11377.
29. Chappell, J. D., Prota, A. E., Dermody, T. S. and Stehle, T. (2002) Crystal structure of reovirus attachment protein sigma1 reveals evolutionary relationship to adenovirus fiber. Embo J, 21: 1-11.
30. Barton, E. S., et al. (2001) Junction adhesion molecule is a receptor for reovirus. Cell, 104: 441-451.
31. Leone, G., et al. (1991) The N-terminal heptad repeat region of reovirus cell attachment protein sigma 1 is responsible for sigma 1 oligomer stability and possesses intrinsic oligomerization function. Virology, 182: 336-345.
32. Leone, G., Mah, D. C. and Lee, P. W. (1991) The incorporation of reovirus cell attachment protein sigma 1 into virions requires the N-terminal hydrophobic tail and the adjacent heptad repeat region. Virology, 182: 346-350.
33. Mah, D.C., Leone, G., Jankowski, J. M. and Lee, P. W. (1990) The N-terminal quarter of reovirus cell attachment protein sigma 1 possesses intrinsic virion-anchoring function. Virology, 179: 95-103.
34. Nakamura, T., et al. (2005) Rescue and propagation of fully retargeted oncolytic measles viruses. Nat Biotechnol, 23: 209-214.
35. Douglas, J. T., et al. (1999) A system for the propagation of adenoviral vectors with genetically modified receptor specificities. Nat Biotechnol, 17: 470-475.
36. Mitraki, A., et al. (1999) Unfolding studies of human adenovirus type 2 fibre trimers. Evidence for a stable domain. Eur J Biochem, 264: 599-606.
37. Leone, G., Maybaum, L. and Lee, P. W. (1992) The reovirus cell attachment protein possesses two independently active trimerization domains: basis of dominant negative effects. Cell, 71: 479-488.
38. Mercier, G. T., et al. (2004) A chimeric adenovirus vector encoding reovirus attachment protein sigma1 targets cells expressing junctional adhesion molecule 1. Proc Natl Acad Sci USA, 101: 6188-6193.
39. Tsuruta, Y., et al. (2005) Reovirus sigma1 fiber incorporated into adenovirus serotype 5 enhances infectivity via a CAR-independent pathway. Biochem Biophys Res Commun, 335: 205-214.
40. Von Seggern, D. J., Kehler, J., Endo, R. I. and Nemerow, G. R. (1998) Complementation of a fibre mutant adenovirus by packaging cell lines stably expressing the adenovirus type 5 fibre protein. J Gen Virol, 79: 1461-1468.
41. DuBridge, R. B., et al. (1987) Analysis of mutation in human cells by using an Epstein-Barr virus shuttle system. Mol Cell Biol, 7: 379-387.
42. Toes, R. E., et al. (1997) Protective anti-tumor immunity induced by vaccination with recombinant adenoviruses encoding multiple tumor-associated cytotoxic T lymphocyte epitopes in a string-of-beads fashion. Proc Natl Acad Sci USA, 94: 14660-14665.
43. Havenga, M. J., et al. (2001) Improved adenovirus vectors for infection of cardiovascular tissues. J Virol, 75: 3335-3342.
44. He, T. C., et al. (1998) A simplified system for generating recombinant adenoviruses. Proc Natl Acad Sci USA, 95: 2509-2514.
45. Carette, J. E., et al. (2005) Replication-dependent transgene expression from a conditionally replicating adenovirus via alternative splicing to a heterologous splice-acceptor site. J Gene Med, 7: 1053-1062.
46. Burstin, S. J., Spriggs, D. R. and Fields, B. N. (1982) Evidence for functional domains on the reovirus type 3 hemagglutinin. Virology, 117: 146-155.
47. Douglas, J. T., et al. (1996) Targeted gene delivery by tropism-modified adenoviral vectors. Nat Biotechnol, 14: 1574-1578.
48. Lichtenstein, D. L., et al. (2004) Functions and mechanisms of action of the adenovirus E3 proteins. Int. Rev. Immunol. 23:75-111.
49. Yu, D.-C., et al. (1999) The addition of adenovirus type 5 region E3 enables calydon virus 787 to eliminate distant prostate tumor xenografts. Cancer Res. 59:4200-4203.
50. Cichon, G., et al. (2003) Titer determination of Ad5 in blood: a cautionary note. Gene Ther 10:1012-1017.
51. Nicol, C. G., et al. (2004) Effect of adenovirus serotype 5 fiber and penton modifications on in vivo tropism in rats. Mol Ther 10:344-354.
52. Lyons, M., et al. (2006) Adenovirus Type 5 Interactions with Human Blood Cells May Compromise Systemic Delivery. Mol Ther. 14:118-128.

### BRIEF DESCRIPTION OF THE DRAWINGS

FIG. 1. Schematic representation of adenovirus fiber (serotype 5), reovirus σ1 (type 3 Dearing), and the fiber-σ1 fusion protein Tail-T(ii)-MH. The fiber molecule contains three regions: the N-terminal tail domain, the shaft domain, and the C-terminal knob domain. The σ1 molecule contains five regions: the T(i), T(ii), T(iii), and T(iv) domains that form the fibrous tail and the C-terminal head domain. The fiber-σ1 fusion protein Tail-T(ii)-MH consists of the adenovirus' fiber tail domain, the reovirus' σ1T(ii) domain, a Myc- and a His tag. Consequently, this fusion lacks the CAR and HSG binding site residing in fiber and the JAM-A and sialic-acid binding site residing in al. The numbers of relevant amino acids and the location of functional regions are indicated. The predicted molecular weights (MW) are shown in kDa. NLS: nuclear localization signal, SA: sialic acid.

FIG. 2. Analysis of trimerization efficiency and nuclear localization of the fiber-σ1 fusion protein. (A) Native (N) and denatured (D) cell lysates of 293T cells transfected with plasmids encoding fiber or Tail-T(ii)-MH were resolved by SDS-PAGE and analysed by immunoblotting using fiber tail-specific MAb Ab4. Molecular weight markers (M) are indicated in kDa. (B) Immunofluorescence of 293T cells transfected with plasmids encoding σ1, fiber, and Tail-T(ii)-MH. The left panels show protein staining detected by using the σ1 head-specific MAb 9BG5 for reovirus σ1 and MAb Ab4 for the other proteins. The right panels show nuclear staining of the same cells detected by using Hoechst 33342.

FIG. 3. Viral spread in time of AdG.L., AdG.L.ΔE3.Tail-T(ii)-MH and AdG.L.Tail-T(ii)-MH. The helper cells 293.HissFv.rec are infected with the indicated AdVs at an MOI of 0.01. Viral spread was visualized by means of GFP expression. Each image is a representative picture of the structures seen in the wells. Numbers represent the days post infection.

FIG. 4. Propagation efficiency of AdG.L, AdG.L.ΔE3.Tail-T(ii)-MH and AdG.L.Tail-T(ii)-MH following infection of 293.HissFv.rec cells. (A) Cells were infected with either AdG.L, AdG.L.ΔE3.Tail-T(ii)-MH or AdG.L.Tail-T(ii)-MH at an MOI of 0.01 IU/cell. At the indicated times after infection, luciferase expression was assessed and presented as percentage of the luciferase value 24 h post infection. (B) Cells were infected with either AdG.L or AdG.L.Tail-T(ii)-MH at an MOI of 0.004 IU/cell. At the indicated times after infection, luciferase expression was assessed as an indicator of AdV propagation. The results are expressed as the average values of an experiment performed in triplicate. Error bars indicate standard deviations.

FIG. 5. Incorporation of Tail-T(ii)-MH into the adenovirus capsid. CsCl-purified particles of AdG.L.Tail-T(ii)-MH or a wild-type fiber-containing adenovirus were denaturated and resolved by SDS-PAGE. (A) Capsid proteins of 1.2×10¹¹ particles were visualized by staining with Coomassie blue. The arrow indicates the location of the Tail-T(ii)-MH fusion protein. (B) Purified particles (5×10⁹) were resolved by SDS-PAGE and transferred to PVDF membranes. Blots were incubated with either tail-specific MAb (left panel) or Myc-specific MAb (right panel), and protein bands were visualized using ECL plus. Molecular weights in kDa of marker proteins are indicated (M).

FIG. 6. De-targeting effect of AdG.L.Tail-T(ii)-MH. Infection efficiency of AdG.L.Tail-T(ii)-MH was analysed using the non-target cell line 293 and the target cell line 293.HissFv.rec. Both cell lines were infected with either AdG.L or AdG.L.Tail-T(ii)-MH at an MOI of 0.5 IU/cell. Following 48 h incubation, transduction efficiency was evaluated by (A) analysis of GFP expression using fluorescence microscopy and (B) measurement of luciferase expression using a chemiluminescence assay. The averaged luciferase activity of three independent experiments is presented as percentage of the activity found after infection of 293HissFv.rec cells. Error bars indicate standard deviations.

FIG. 7. Analysis of the infection specificity of AdG.L.Tail-T(ii)-MH. (A) AdG.L and AdG.L.Tail-T(ii)-MH were incubated in the presence or absence of 300 ng of knob-specific MAb or His-specific MAb at room temperature for 2 h prior to infection of either 293 or 293.HissFv.rec cells at an MOI of 0.5 IU/cell. (B) The cell lines CHO and CHO-αHis were infected with AdG.L or AdG.L.Tail-T(ii)-MH at an MOI of 5 IU/cell. Following 48 h incubation, transduction efficiency was assessed by luciferase expression. The results are expressed as the average luciferase activity for three experiments. Error bars indicate standard deviations.

FIG. 8. Persistence of AdG.L.p*Tail-T(ii)-MH in the circulation of C57bl/6 mice. A dose of 1E+10 vp of AdG.L or AdG.L.p*Tail-T(ii)-MH was administered intravenously into C57bl/6 mice (n=5 for AdG.L.p*Tail-T(ii)-MH and n=6 for AdG.L). At 2, 5, 10, 20, 30, 60, and 120 minutes after administration, blood samples were taken and the titer of infectious virus in each sample was determined by means of the luciferase expression after infecting 293.HissFv.rec cells. A dilution series of each AdV was used as standard of luciferase expression.

FIG. 9

Amino acid sequence of Tail-T(ii)-MH:

FIG. 10

Amino acid sequence of Tail-T(ii)ev-Ang:

FIG. 11

Amino acid sequence of Tail-T(ii)ev-CD40L:

FIG. 12. Interaction of AdG.L, AdG.L.Tail-T(ii)-MH and AdG.L.p*Tail-T(ii)-MH with human erythrocytes. (A) Hemagglutination of AdVs and human erythrocytes. A suspension of 1% packed erythrocytes was gently mixed with an equal volume of virus dilutions as indicated (or with buffer without virus; control) and left to sediment before hemagglutination was evaluated. (B) Association of AdVs with human red blood cells measured by real time PCR. AdVs (3.4×10⁹ vp/ml) were incubated with a physiologic concentration of washed human erythrocytes in PBS at 37° C. After 60 minutes incubation, the cellular (bound) and supernatant (unbound) fractions were separated by centrifugation. The cellular fraction was washed twice with 10 volume equivalents PBS, before viral genomes present in each fraction were quantified by real time PCR. The results are presented as average percentage recovered in each fraction from three independent red blood cell donors. Error bars represent standard deviations.

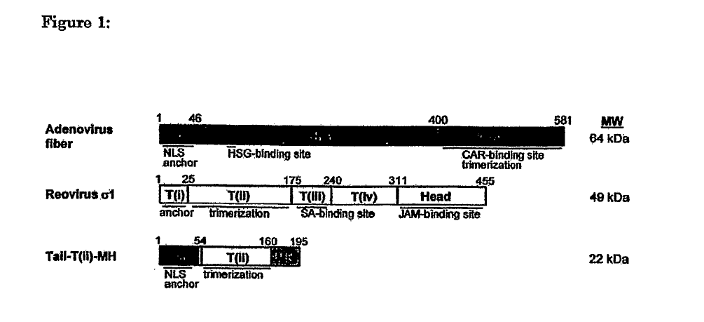

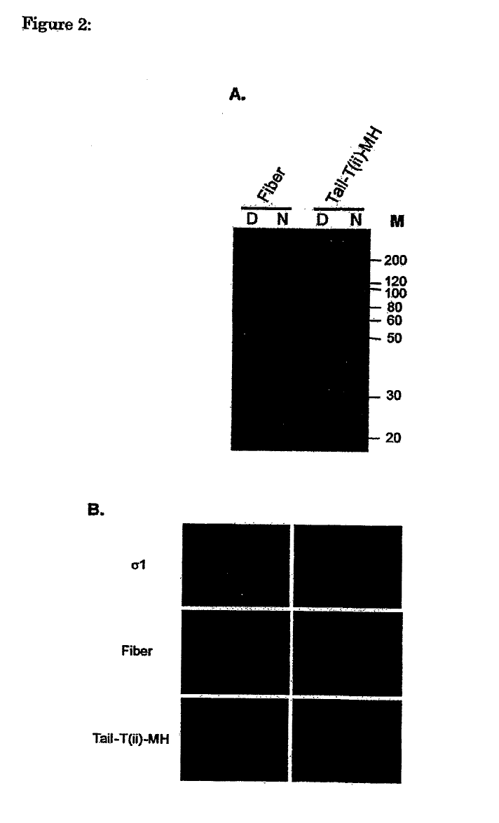

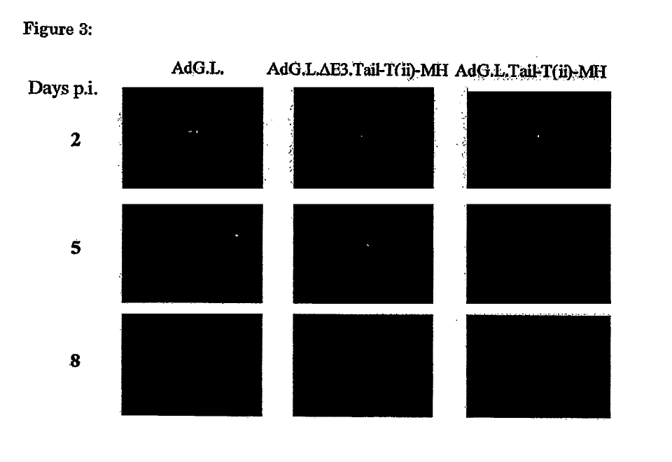

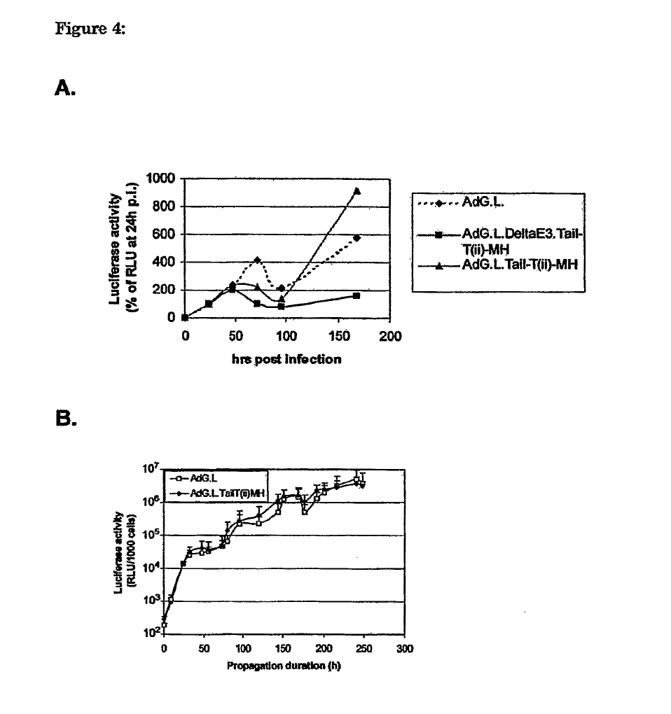

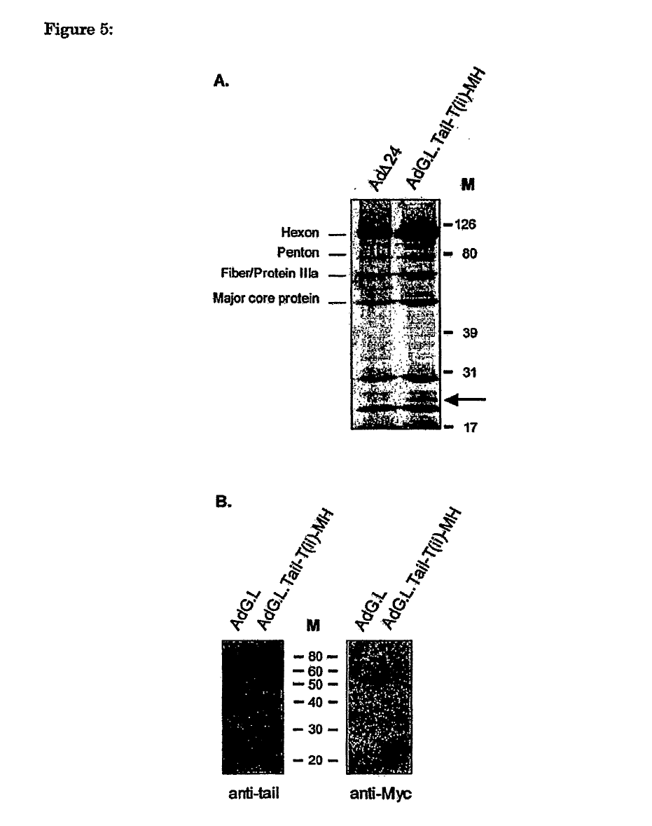

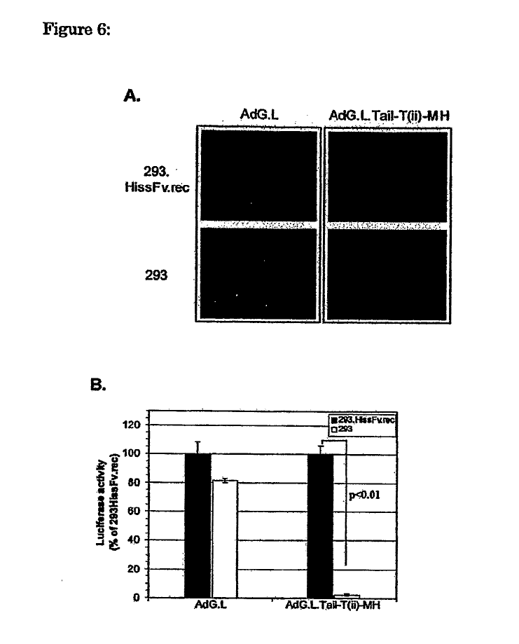

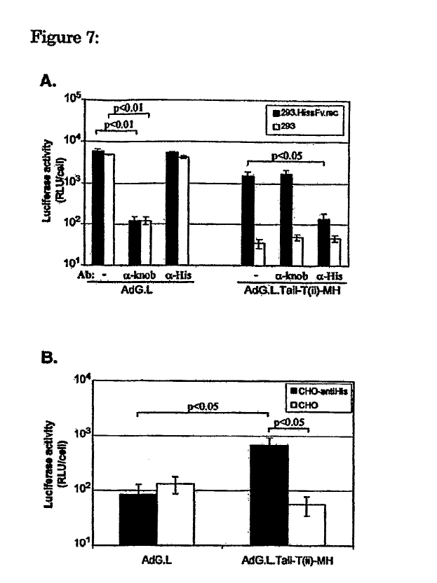

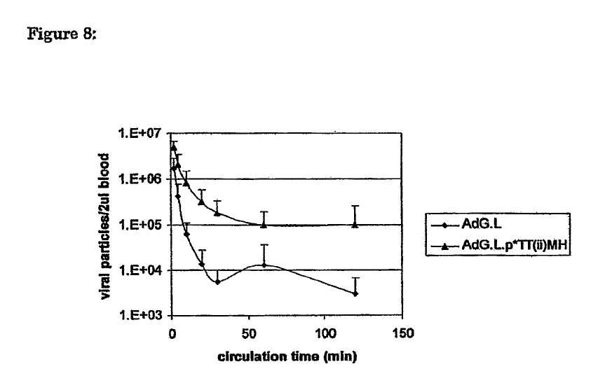

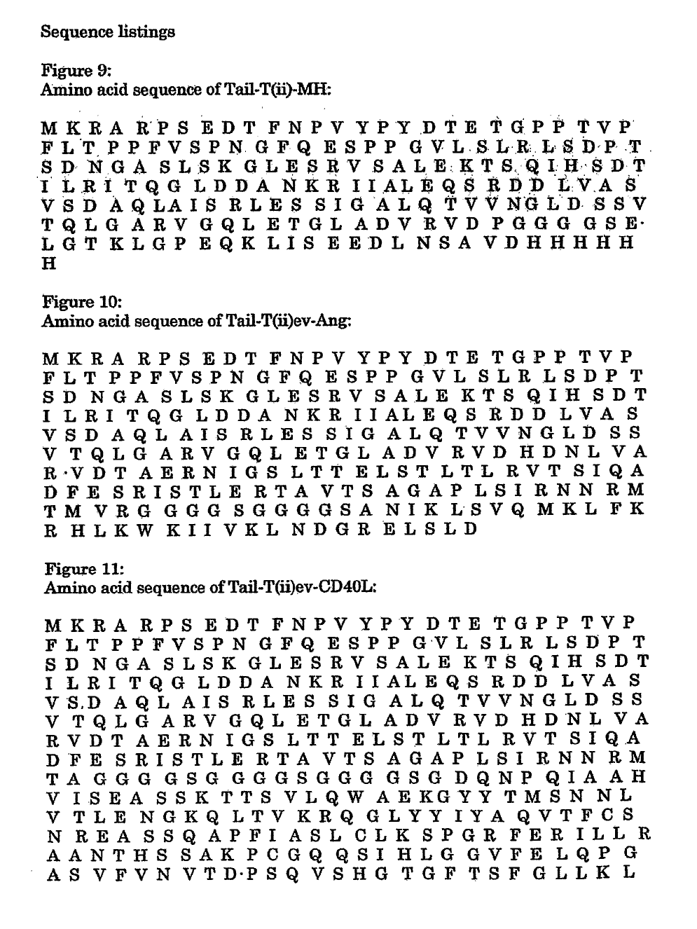

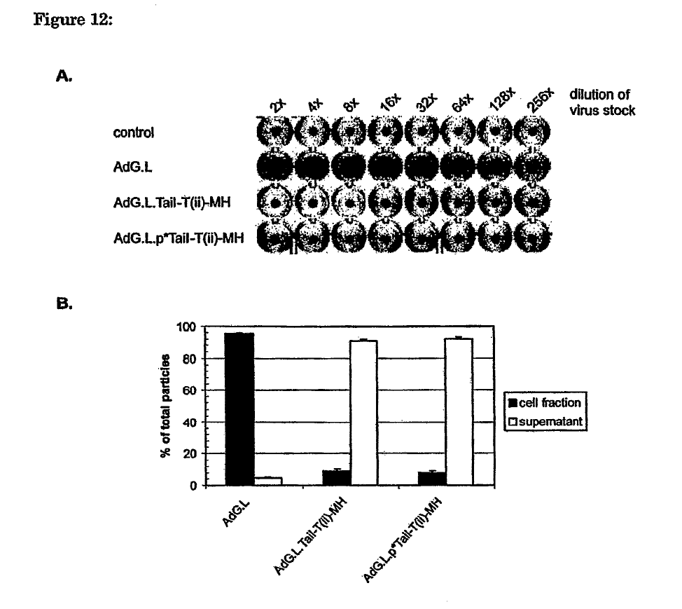

TABLE 1
Yields of crude batches of three different AdV that were produced by
infecting semi-confluent monolayers of E1-complementing packaging
cells in a 182 cm² culture flask.

| | Genome-containing particles |
|---|---|
| Virus | per flask |
| AdG.L | 7.9 × 10⁹ |
| AdG.L.ΔE3.Tail-T(ii)-MH | 6.0 × 10⁸ |
| AdG.L.Tail-T(ii)-MH | 5.4 × 10⁹ |

1. An adenovirus particle comprising nucleic acid derived from an adenovirus and comprising a chimeric adenovirus spike protein, wherein said spike protein essentially lacks a functional knob domain and comprises an oligomerization domain of reovirus attachment protein σ1 or a functional part, derivative and/or analogue thereof, and wherein said nucleic acid comprises at least one coding region for a protein of an adenovirus early region 3 (E3) region or a functional part, derivative and/or analogue of said E3 protein.

2. An adenovirus particle according to claim 1, wherein said nucleic acid further comprises at least one coding region for said chimeric adenovirus spike protein.

3. An adenovirus particle according to claim 1 or 2, wherein said oligomerization domain comprises a reovirus σ1 T(ii) domain or a functional part, derivative and/or analogue thereof.

4. An adenovirus particle according to any one of claims 1-3, wherein said chimeric adenovirus spike protein comprises an adenovirus fiber tail domain or a functional part, derivative and/or analogue thereof.

5. An adenovirus particle according to claim 4, wherein said adenovirus fiber tail domain or a functional part, derivative and/or analogue thereof and said reovirus σ1 T(ii) domain or a functional part, derivative and/or analogue thereof are separated by a hinge region, preferably a hinge region derived from reovirus σ1 protein.

6. An adenovirus particle according to any one of claims 1-5, comprising a recombinant adenovirus virus vector.

7. An adenovirus particle according to claim 6, wherein said adenovirus vector comprises a nucleic acid with a coding region for a gene of interest, preferably a therapeutic protein.

8. An adenovirus particle according to any one of claims 1-7, further comprising nucleic acid encoding p53 or a functional part, derivative, analogue or mutant thereof.

9. An adenovirus particle according to any one of claims 1-8, comprising nucleic acid encoding an adenovirus E1 region protein or a functional part, derivative and/or analogue thereof.

10. An adenovirus particle according to any one of claims 1-9, comprising nucleic acid derived from an adenovirus that encodes a replication competent adenovirus.

11. An adenovirus particle according to claim 10, wherein nucleic acid encoding said replication competent adenovirus comprises an adaptation for preferential replication of said replication competent adenovirus in a transformed cell when compared to an untransformed cell of otherwise the same type.

12. An adenovirus particle according to claim 11, wherein said adaptation comprises a nucleic acid comprising a coding region encoding an adenovirus E1A protein wherein said E1A protein comprises a mutation in at least part of the pRb-binding CR2 domain, preferably a deletion encompassing amino acids 122 to 129 (LTCHEAGF) of E1A.

13. A nucleic acid comprising a coding region for a chimeric adenovirus spike protein that essentially lacks a functional knob domain and comprises an oligomerization domain of reovirus attachment protein σ1 or a functional part, derivative and/or analogue thereof and wherein said nucleic acid further comprises at least one coding region of an adenovirus E3 region protein or a functional part, derivative and/or analogue thereof.

14. A method for producing an adenovirus comprising providing a host cell that is permissive for replication of said adenovirus with an adenovirus particle according to any one of claims 1-12, or a nucleic acid according to claim 13.

15. An isolated and/or recombinant cell comprising a nucleic acid according to claim 13.

16. A method for providing nucleic acid to a cell comprising contacting said cell with an adenovirus virus particle according to any one of claims 1-12.

17. A composition comprising adenovirus particles according to any one of claims 1-12.

18. A composition comprising adenovirus particles comprising a chimeric adenovirus spike protein that essentially lacks a functional knob domain and comprises an oligomerization domain of reovirus attachment protein σ1 or a functional part, derivative and/or analogue thereof, wherein said composition is essentially free of protein that contains an essentially functional knob domain.

19. A composition comprising adenovirus particles comprising a chimeric adenovirus spike protein, obtainable by a method according to claim 14.

20. A method for preparing a composition comprising an adenovirus particle that comprises a chimeric adenovirus spike protein that essentially lacks a functional knob domain and comprises an oligomerization domain of reovirus attachment protein σ1 or a functional part, derivative and/or analogue thereof, said method comprising providing cells that are permissive for adenovirus replication with an adenovirus vector, with nucleic acid encoding said chimeric adenovirus spike protein and with nucleic acid encoding at least one adenovirus E3 region protein or a functional part, derivative and/or analogue thereof, wherein said permissive cells are essentially lacking protein that contains an essentially functional knob domain, said method further comprising culturing said permissive cells to allow for at least one replication cycle of said adenovirus vector and harvesting said adenovirus particle.

21. A composition comprising adenovirus particles comprising a chimeric adenovirus spike protein that essentially lacks a functional knob domain obtainable by a method according to claim 20.

22. A composition according to claim 21, essentially free of protein that contains an essentially functional knob domain.

23. A purified adenovirus particle composition according to claim 21 or claim 22, comprising essentially similar amounts of co-purified contaminants as a similarly purified preparation of a comparable adenovirus comprising adenovirus fiber protein that contains an essentially functional knob domain.

24. A method for providing an individual with an adenovirus particle comprising administering to said individual an adenovirus particle according to any one of claims 1-12 or a composition according to any one of claims 17-19, 21-23.

25. A method according to claim 24, for the treatment of a disease in said individual.

26. Use of an adenovirus particle according to any one of claims 1-12 or a composition according to any one of claims 17-19, 21-23 for the preparation of a medicament and/or vaccine.

27. A method for preparing a composition comprising an adenovirus particle according to claim 20, wherein said cells are stably transformed with nucleic acid encoding at least one E3 protein or a functional part, derivative and/or analogue thereof.

28. A method according to claim 27 wherein expression of said E3 region protein is inducible.

29. An adenovirus particle according to any one of claims 1-12, wherein said chimeric adenovirus spike protein further comprises a binding moiety.

30. A nucleic acid according to claim 13, wherein said chimeric adenovirus spike protein further comprises a binding moiety.

31. An adenovirus vector comprising a chimeric adenovirus spike protein that essentially lacks a functional knob domain and comprises an oligomerization domain of reovirus attachment protein σ1 or a functional part, derivative and/or analogue thereof, said vector further comprising a coding region for p53 protein.

32. An adenovirus particle according to any one of claims 1-12, comprising an expression cassette comprising said coding region for an E3 protein or functional part, derivative and/or analogue thereof.

33. An adenovirus particle according to claim 32, wherein said expression cassette comprises a heterologous promoter and/or heterologous splice site.

34. An adenovirus particle according to claim 29, wherein said binding moiety comprises a peptide derived from CD40.

35. An adenovirus particle according to claim 29, wherein said binding moiety comprises Anginex.

36. Use of a chimeric adenovirus spike protein that essentially lacks a functional knob domain and comprises an oligomerization domain of reovirus attachment protein σ1 or a functional part, derivative and/or analogue thereof, for producing an adenovirus particle.

37. Use according to claim 36, for producing an adenovirus particle that exhibits reduced binding to a red blood cell when compared to an adenovirus particle comprising a functional knob domain.

38. Use of an oligomerization domain of reovirus attachment protein σ1 or a functional part, derivative and/or analogue thereof for producing an adenovirus particle that exhibits reduced binding to a red blood cell when compared to an adenovirus particle comprising a functional knob domain.

39. A composition comprising an adenovirus particle comprising a chimeric adenovirus spike protein that essentially lacks a functional knob domain and comprises an oligomerization domain of reovirus attachment protein σ1 or a functional part, derivative and/or analogue thereof, and a red blood cell.

40. A composition according to claim 29, wherein said red blood cell is a human red blood cell.
